(19) United States
(12) Patent Application Publication
(10) Pub. No.: US 2011/0041220 A1
(43) Pub. Date: Feb. 17, 2011

(54) MUTANT GENE THAT REGULATES OIL-AND-FAT CONTENT IN SEED AND METHOD FOR REGULATING OIL-AND-FAT CONTENT IN SEED

(75) Inventors: Chikara Ohto, Toyota-shi (JP); Norihiro Mitsukawa, Miyoshi-shi (JP); Makoto Hayashi, Toyohashi-shi (JP)

Correspondence Address: SUGHRUE MION, PLLC, 2100 PENNSYLVANIA AVENUE, N.W., SUITE 800, WASHINGTON, DC, 20037, US

(73) Assignee: TOYOTA JIDOSHA KABUSHIKI KAISHA, Toyota-shi, Aichi (JP)

(21) Appl. No.: 12/920,063

(22) PCT Filed: Feb. 23, 2009
(86) PCT No.: PCT/JP2009/053784

§ 371 Date: Aug. 27, 2010

(30) Foreign Application Priority Data
Feb. 28, 2008 (JP) 2008-048612

(51) Int. Cl. A01H 5/00; C07H 21/00; C07K 14/415; C12N 5/10

(52) U.S. Cl. 800/298; 536/23.6; 530/370; 435/419

(57) ABSTRACT

This invention provides a mutant gene having the function of regulating the oil-and-fat content in seeds and a method for regulating the oil-and-fat content in seeds. The mutant gene encodes a mutant protein having a consensus sequence resulting from substitution of the 12th amino acid residue from the N-terminus of the amino acid sequence as shown in SEQ ID NO: 1 with another amino acid and having the function of regulating the oil-and-fat content in seeds.

### TECHNICAL FIELD

The present invention relates to a mutant gene that regulates the oil-and-fat content in seeds, which has been identified using changes in oil bodies existing in seeds as an indicator, and a method for regulating the oil-and-fat content in seeds through mutation of a given gene.

### BACKGROUND ART

Oil bodies (occasionally referred to as “lipid bodies”) are organelles that are present in large quantities in plants, and particularly in seed cells of oil crops. Oil bodies comprise a monolayer membrane of phospholipid comprising specific proteins referred to as oleosins, steroleosins, and caleosins, and they accumulate plant oil-and-fats in the form of triacylglycerol (TAG, neutral fat, or neutral lipid) therein. In particular, large quantities of plant fats are accumulated in plant seeds. In the past, oil-and-fats accumulated in oil bodies have been analyzed by methods that involve pulverizing seeds to extract oil-and-fat components and performing gas chromatography, liquid chromatography, or other techniques. Such analytical methods, however, required the addition of lipid degradation inhibitors and processing at low temperatures. In addition, oil-and-fat components may be disadvantageously degraded.

Siloto, R. M. P. et al., Plant Cell 18, 1961-1974, 2006 discloses that oil body size is influenced by oleosin quantity. Wahlroos et al., GENESIS, 35 (2): 125-132, 2003 discloses that the oleosin gene is fused to the green fluorescent protein (GFP) gene and oil bodies can be observed with the aid of GFP fluorescence. Even if it is possible to observe oil bodies, however, the correlation between the number or form of oil bodies and the oil-and-fat amount or type accumulated in oil bodies has not yet been elucidated.

### DISCLOSURE OF THE INVENTION

The present invention is intended to establish a system that allows measurement of various properties, such as the number or size of oil bodies in plant cells. Such system elucidates the correlation between properties of oil bodies and the oil-and-fat content. The present invention is also intended to provide a mutant gene having the function of regulating the oil-and-fat content in seeds and a method for regulating the oil-and-fat content in seeds.

The present inventors have conducted concentrated studies in order to attain the above objects, expressed an oleosin-GFP fusion protein, and discovered a positive correlation between the sum of GFP fluorescence intensities and oil-and-fat content. Based on such findings, the present inventors discovered a gene having the function of influencing the oil-and-fat content in seeds and the influence of a mutation of an amino acid substitution in such gene on the oil-and-fat content in seeds, thereby completing the present invention.

Specifically, the mutant gene of the present invention includes the following.

(1) A mutant gene encoding a mutant protein having a consensus sequence derived from the amino acid sequence as shown in SEQ ID NO: 1 by substitution of the 12th amino acid residue from the N terminus with another amino acid and having the function of regulating the oil-and-fat content in plant seeds.

(2) The mutant gene according to (1), wherein the other amino acid is an amino acid selected from the group consisting of isoleucine, valine, leucine, and methionine.

(3) The mutant gene according to (1), wherein the other amino acid is isoleucine.

(4) The mutant gene according to (1), wherein the mutant protein has a sequence derived from the consensus sequence included in an amino acid sequence as shown in any one of SEQ ID NOs: 4 to 17 by substitution of the 12th amino acid residue from the N terminus with another amino acid.

(5) The mutant gene according to (1), which is a mutant of a plant-derived gene.

(6) The mutant gene according to (1), which is a mutant of a gene derived from a plant belonging to a family selected from the group consisting of Gramineae, Brassicaceae, and Salicaceae.

(7) The mutant gene according to (1), which is a mutant of a gene derived from a plant belonging to a species selected from the group consisting of barley, rice, Arabidopsis thaliana, Brassica rapa, and Populus nigra.

In addition, the present invention includes a mutant protein encoded by the mutant gene mentioned above, a transgenic cell into which the mutant gene has been introduced, and a transgenic plant into which the mutant gene has been introduced.

The methods for regulating the oil-and-fat content in seeds of the present invention include the following.

(8) A method for regulating the oil-and-fat content in seeds comprising introducing a mutation in which the 12th amino acid residue from the N terminus of the amino acid sequence as shown in SEQ ID NO: 1 is substituted with another amino acid into a gene encoding a protein having a consensus sequence comprising the amino acid sequence as shown in SEQ ID NO: 1.

(9) The method for regulating the oil-and-fat content in seeds according to (8), wherein the other amino acid is an amino acid selected from the group consisting of isoleucine, valine, leucine, and methionine.

(10) The method for regulating the oil-and-fat content in seeds according to (8), wherein the other amino acid is isoleucine.

(11) The method for regulating the oil-and-fat content in seeds according to (8), wherein a mutant protein resulting from substitution of the 12th amino acid residue from the N terminus with another amino acid in the consensus sequence included in an amino acid sequence as shown in any one of SEQ ID NOs: 4 to 17 is expressed.

(12) The method for regulating the oil-and-fat content in seeds according to (8), wherein a gene derived from a plant belonging to a family selected from the group consisting of Gramineae, Brassicaceae, and Salicaceae is varied.

(13) The method for regulating the oil-and-fat content in seeds according to (8), wherein a gene derived from a plant belonging to a species selected from the group consisting of barley, rice, Arabidopsis thaliana, Brassica rapa, and Populus nigra is varied.

This description includes part or all of the contents as disclosed in the description and/or drawings of Japanese Patent Application No. 2008-048612, which is a priority document of the present application.

### BRIEF DESCRIPTION OF THE DRAWINGS

FIG. 1 schematically shows the amino acid substitution scoring matrix (BLOSUM).

FIG. 2-1 is an alignment diagram showing the results of alignment analysis of 14 types of amino acid sequences.

FIG. 2-2 is an alignment diagram showing the results of alignment analysis of 14 types of amino acid sequences.

FIG. 2-3 is an alignment diagram showing the results of alignment analysis of 14 types of amino acid sequences.

FIG. 2-4 is an alignment diagram showing the results of alignment analysis of 14 types of amino acid sequences.

FIG. 3 shows a molecular evolution dendrogram obtained from the alignment diagram shown in FIG. 2.

FIG. 4A schematically shows the constitution of the oleosin-GFP fusion gene, and FIGS. 4B to 4D each show a fluorescence picture of cotyledons of OleG, mutant A, and mutant B 6 days after the initiation of germination in the dark.

FIG. 5 is a characteristic diagram showing the correlation of the sum of GFP fluorescence (%) and the oil-and-fat content in seeds.

FIG. 6 is a characteristic diagram showing the results of analysis of fatty acid compositions of seed reserved lipids of OleG, mutant A, and mutant B.

FIG. 7 is a photograph showing the results of SDS-PAGE using protein samples extracted from seeds of wild-type Arabidopsis thaliana, OleG, mutant A, and mutant B, the results of immunoblot analysis using an anti-oleosin antibody, and the results of immunoblot analysis using an anti-GFP antibody (C).

FIG. 8 is a photograph showing the results of electron microscopic observation of seed cells of OleG, mutant A, and mutant B.

FIG. 9A is a characteristic diagram showing the results of high-precision mapping for identifying a causal gene of mutant A and FIG. 9B schematically shows the structure of the gene (At1g54570) that is present in the mapped position.

### BEST MODES FOR CARRYING OUT THE INVENTION

Hereafter, the present invention is described in detail with reference to the drawings.

In the present invention, an oil body-specific protein and a fluorescent protein (more particularly the oleosin-GFP fusion gene) were used to transform a model plant Arabidopsis thaliana, and oil bodies contained in the cotyledons obtained from the transgenic Arabidopsis thaliana plant were fluorescently visualized. A mutation was induced in such transgenic Arabidopsis thaliana plant, changes in various properties, such as the configuration and the number of oil bodies, were observed, and changes in the oil-and-fat content and oil compositions were measured. Surprisingly, a positive correlation between the fluorescence sum in cotyledons (i.e., fluorescence intensity per unit area) among various properties of oil bodies and the oil-and-fat content in seeds was found. Based on such finding, a causal gene for a mutant that changes in the oil-and-fat content, the oil-and-fat amount, and the fatty acid composition in the oil-and-fat in a plant and a causative mutation were identified.

Specifically, the mutant gene of the present invention encodes a protein resulting from substitution of a given amino acid in the evolutionarily conserved amino acid sequence referred to as the “diacylglycerol acyltransferase domain” with another amino acid and having the function of regulating the oil-and-fat content and the oil-and-fat amount in a plant. The mutant gene of the present invention is a protein having an evolutionarily conserved amino acid sequence referred to as the diacylglycerol acyltransferase domain comprising an amino acid sequence resulting from substitution of a given amino acid with another amino acid. In the description below, an amino acid sequence constituting a evolutionarily conserved region referred to as the diacylglycerol acyltransferase domain is referred to as the “consensus sequence.” SEQ ID NO: 1 shows the consensus sequence.

Specifically, the consensus sequence shown in SEQ ID NO: 1 can also be represented as the amino acid sequence below (using a single-letter code for the amino acids). (D/E)xxxxxGxxxx(T/S)xxxx(Y/F)(R/K/N/Q)L(F/L)xxxx(F/H)VxL(Y/F)PGGxRE(A/S)LHx( K/R)(G/D)Ex(Y/H)(K/R/Q)L(F/I)WP(D/E)(Q/H/R)xEFVRxA(A/S)(R/K/Q)F(G/N)(A/V/T)(T /K)(I/V)(I/V)PFGxVGEDD(V/F/I/L/M)x(E/D/H)(LN/M/I)x

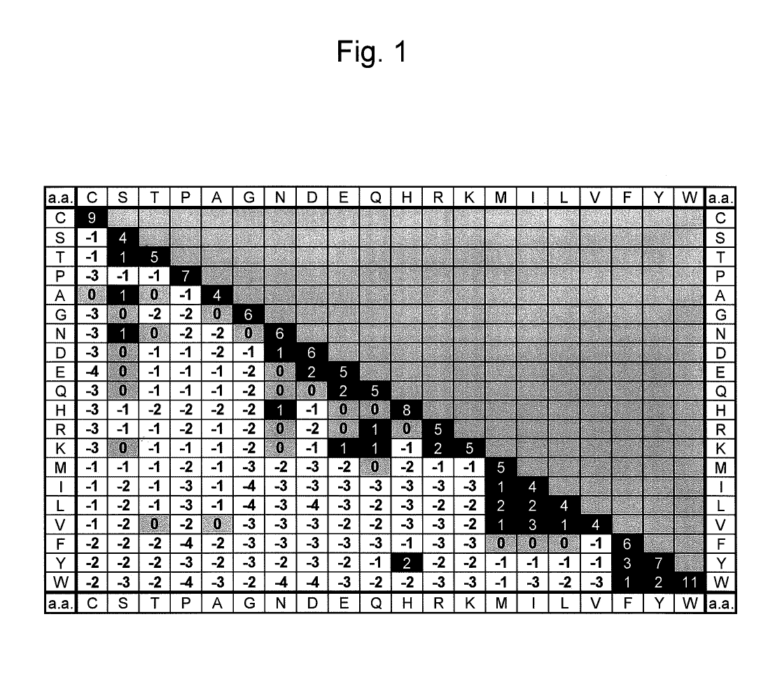

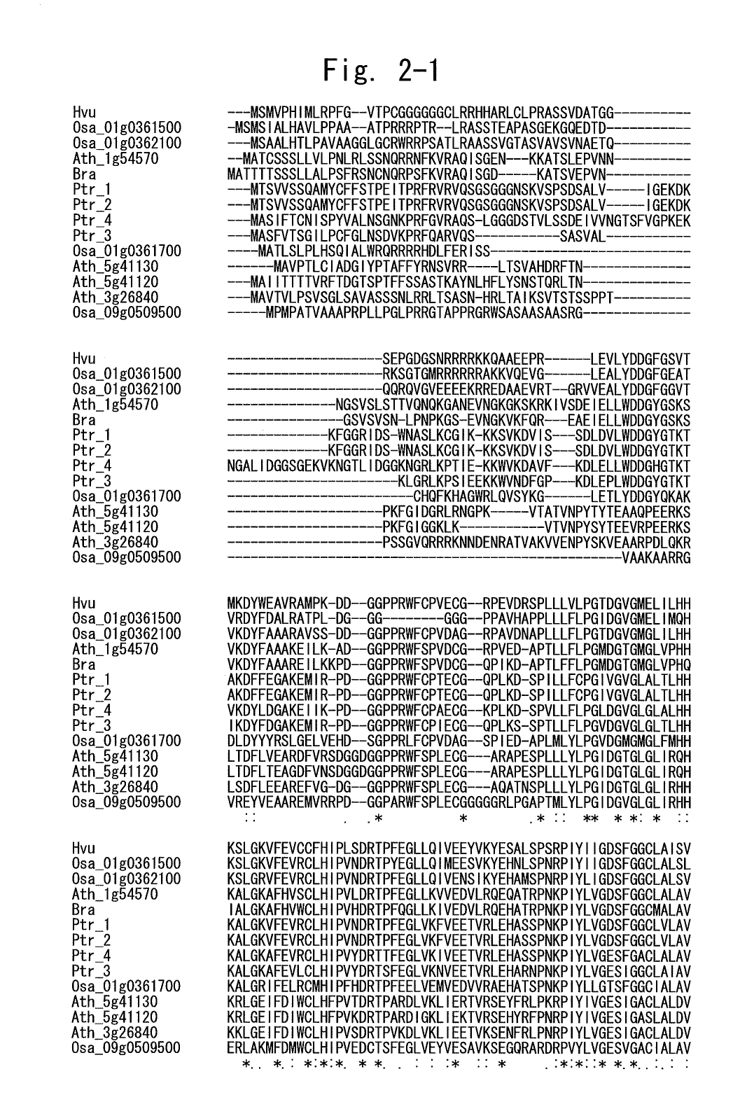

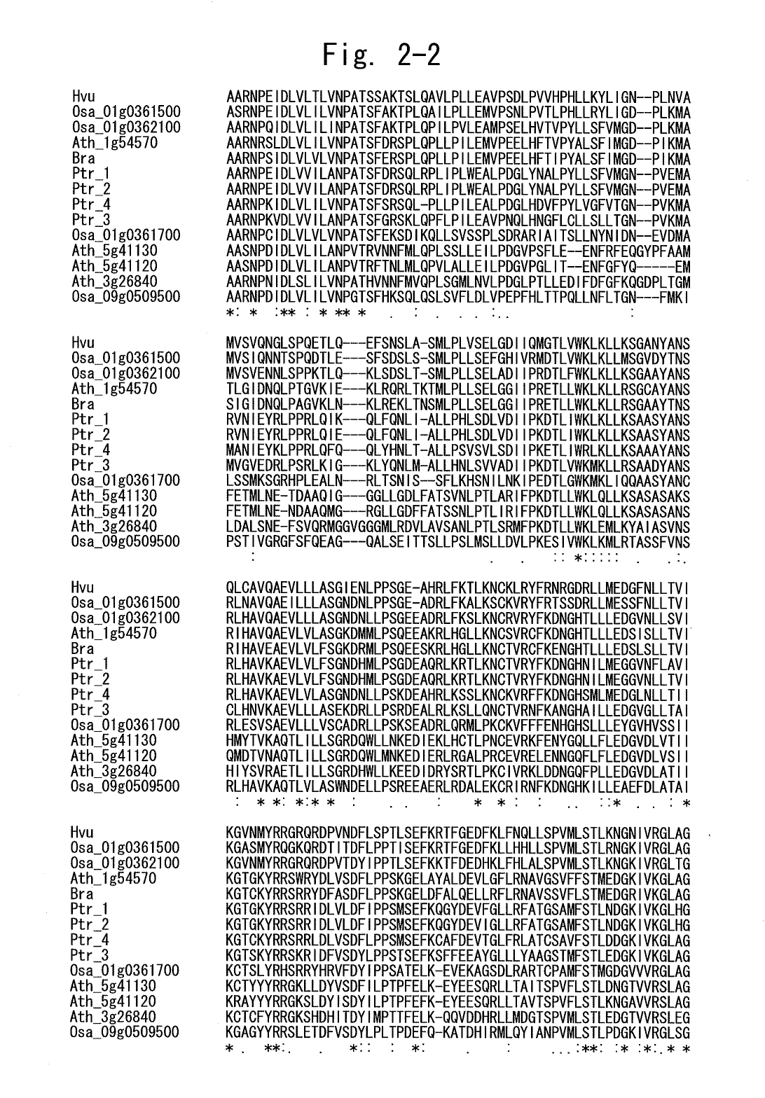

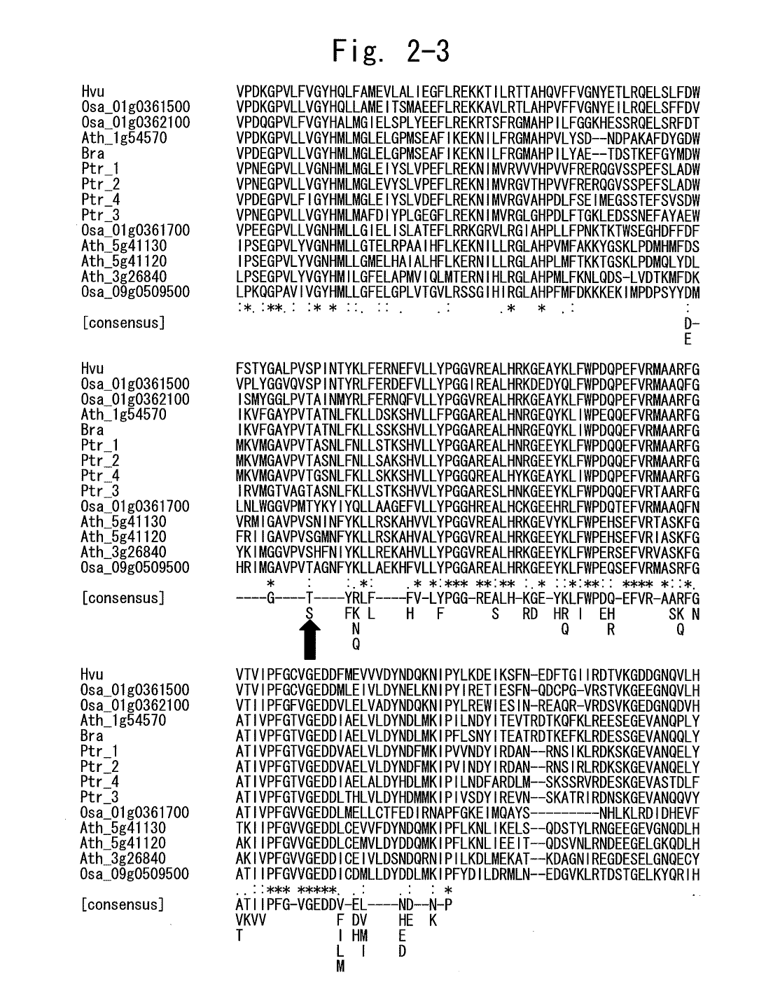

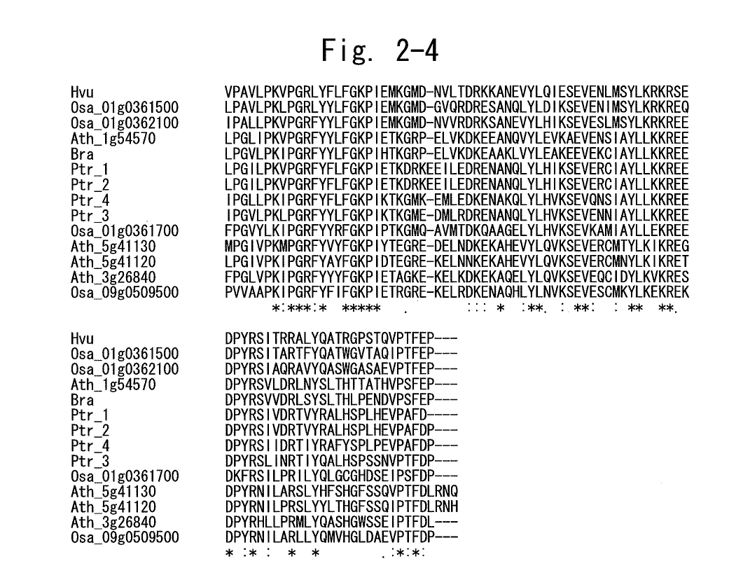

In such amino acid sequence, each plurality of amino acids in parentheses individually represents the variation of an amino acid residue in the position of interest. In the amino acid sequence, x signifies that arbitrary amino acid residues can be used at such position. Such variation of amino acid residues that can be used in a given position occurs for the following reasons. Specifically, the aforementioned consensus sequence can be normalized by subjecting the amino acid sequences of homologous proteins derived from a plurality of plant species to multiple alignment analysis. As described in Reference (1) “McKee's Biochemistry,” Vol. 3, Chap. 5, Amino acid, Peptide, Protein, 5.1 Amino acid, Atsushi Ichikawa (editor), Shinichi Fukuoka (translator), Ryosuke Sone (publisher), published by Kagaku-Dojin Publishing Co., Inc., ISBN4-7598-0944-9, it is well-known that amino acids are classified in accordance with side chains having similar properties (i.e., chemical properties or physical size). Also, it is well-known that, in molecular evolution, substitution of amino acid residues, which are classified into a given group, takes place at high frequency while protein activity is retained. Based on such concept, Reference (2): Henikoff S., Henikoff J. G, Amino-acid substitution matrices from protein blocks, Proc. Natl. Acad. Sci., U.S.A., 89, 10915-10919, 1992 proposes in its FIG. 2 the amino acid substitution scoring matrix (BLOSUM), and this is extensively used (FIG. 1 of the present description). Reference (2) is based on the principle that substitution of amino acids having similar side chain chemical properties imposes little influence on proteins in terms of structural or functional changes. According to References (1) and (2), side chain groups of amino acids in terms of the multiple alignment can be determined based on indicators such as chemical properties or physical sizes. This is indicated as a group of amino acids having a score of 0 or larger, and preferably as a group of amino acids having a score of 1 or larger, as shown in FIG. 1. Examples of representative groups include the 8 groups shown below. Other more specific classifications may be a group of amino acids having values of 0 or higher, preferably a group of amino acids having values of 1 or higher, and more preferably a group of amino acids having values of 2 or higher, as shown in FIG. 1.

### 1) Group of Hydrophobic Aliphatic Amino Acids (ILMV Group)

This is a group of amino acids having aliphatic hydrophobic side chains selected from among the neutral and non-polar amino acids described in Reference (1), which is composed of V (Val, valine), L (Leu, leucine), I (Ile, isoleucine), and M (Met, methionine). Among amino acids that are classified as neutral and non-polar amino acids according to Reference (1), FGACWP is not included in “the group of aliphatic hydrophobic amino acids” for the following reasons. That is, G (Gly, glycine) and A (Ala, alanine) are smaller than methyl groups and have small non-polar effects. Also, C (Cys, cysteine) occasionally plays a key role in S—S bonds and forms a hydrogen bond with an oxygen or nitrogen atom. Further, side chains of F (Phe, phenylalanine) and W (Trp, tryptophan) have very large molecular weights and have potent aromatic effects. Also, P (Pro, proline) has potent imino acid effects and disadvantageously immobilizes the angle of the polypeptide main chain.

### 2) Group Having Hydroxymethylene Group (ST Group)

This is a group of amino acids having hydroxymethylene groups on the side chains selected from among the neutral and polar amino acids, which is composed of S (Ser, serine) and T (Thr, threonine). Since hydroxyl groups that are present on S and T side chains are sugar-binding sites, such hydroxyl groups often serve as important sites allowing a given polypeptide (a protein) to have a given activity.

### 3) Group of Acidic Amino Acids (DE Group)

This is a group of amino acids having acidic carboxyl groups on the side chains, which is composed of D (Asp, aspartic acid) and E (Glu, glutamic acid).

### 4) Group of Basic Amino Acids (KR Group)

This is a group of basic amino acids, which is composed of K (Lys, lysine) and R (Arg, arginine). K and R positively charge over a wide pH range and have basic properties. H (His, histidine), which is classified as a basic amino acid, is not substantially ionized at pH 7, and thus it is not classified into this group.

### 5) Group of Amino Acids Having Methylene Groups or Polar Groups (DHN Groups)

Amino acids of this group have carbon atoms at the cc positions, methylene groups bound thereto as side chains, and polar groups at farther positions. Physical sizes of non-polar methylene groups are very similar, and the group is composed of N (Asn, asparagine; an amide group as a polar group), D (Asp, aspartic acid; a carboxyl group as a polar group), and H (His, histidine; an imidazole group as a polar group).

### 6) Group of Amino Acids Having Dimethylene Groups or Polar Groups (EKQR Groups)

Amino acids of this group have carbon atoms at the a positions, linear hydrocarbons of dimethylene or higher bound thereto as side chains, and polar groups at farther positions. Physical sizes of non-polar dimethylene groups are very similar, and such groups are composed of E (Glu, glutamic acid; a carboxyl group as a polar group), K (Lys, an amino group as a polar group), Q (Gln, glutamine; an amide group as a polar group), and R (Arg, arginine; imino and amino groups as a polar groups).

### 7) Group of Aromatic Amino Acids (FYW Group)

This is a group of aromatic amino acids having benzene nuclei on the side chains, and this group has chemical properties peculiar to aromatic amino acids. The group is composed of F (Phe, phenylalanine), Y (Tyr, tyrosine), and W (Trp, tryptophan).

### 8) Group of Cyclic and Polar Amino Acids (HY Group)

This group is composed of amino acids simultaneously having a cyclic structure and a polar group on the side chains. The group is composed of H (H, histidine; the cyclic structure and the polar group are imidazole groups) and Y (Tyr, tyrosine; the cyclic structure is the benzene nucleus and the polar group is a hydroxyl group).

The mutant gene of the present invention results from substitution of the 12th amino acid residue from the N terminus in the aforementioned consensus sequence (i.e., threonine (T) or serine (S)) with another amino acid, and it encodes a protein having the function of regulating the oil-and-fat amount in a plant. The 12th amino acid residue from the N terminus in the aforementioned consensus sequence (i.e., threonine (T) or serine (S)) is an amino acid residue that is highly likely to be phosphorylated by threonine kinase or serine kinase, and it is highly likely to be an amino acid residue that remarkably contributes to diacylglycerol acyltransferase activity. By substituting the 12th amino acid residue from the N terminus in the aforementioned consensus sequence (i.e., threonine (T) or serine (S)) with another amino acid, accordingly, the activity of a protein encoded by the gene of the present invention would be changed. The situation in which activity of a protein encoded by the gene of the present invention would be changed refers to a situation in which enzyme activity is improved, enzyme activity is inhibited, substrate specificity is changed, or affinity with a substrate, a coenzyme, or another factor is changed.

Examples of other amino acids after substitution include isoleucine, valine, leucine, and methionine, with isoleucine being particularly preferable. When the 12th amino acid residue from the N terminus in the aforementioned consensus sequence (i.e., threonine (T) or serine (S)) is substituted with isoleucine, the resultant exhibits a characteristic phenotype. That is, the oil-and-fat amount per seed grain is increased; however, the oil-and-fat content (%) per seed grain is decreased in comparison with that of a wild-type plant.

The consensus sequence may be the sequence (shown in SEQ ID NO: 2) resulting from the addition of a sequence comprising 10 amino acid residues (xxx(N/H/E/D)(D/E)xx(N/K)xP) to the C terminus of the amino acid sequence as shown in SEQ ID NO: 1. Specifically, the mutant gene of the present invention results from the substitution of the 12th amino acid residue (threonine (T) or serine (S)) from the N terminus of the consensus sequence resulting from the addition of a given sequence (xxx(N/H/E/D)(D/E)xx(N/K)xP) to the C terminus of the amino acid sequence as shown in SEQ ID NO: 1 with another amino acid, and it encodes a protein having the function of regulating the oil-and-fat amount in a plant.

Genes encoding a protein having the consensus sequence are not limited to genes of given plant species, and such genes can be identified and isolated from a wide variety of plant species. Specifically, genes encoding a protein having the consensus sequence can be identified and isolated from Arabidopsis thaliana of Brassicaceae, barley (Hordeum vulgare) of Gramineae, rice (Oryza sativa) of Gramineae, Brassica rapa of Brassicaceae, and Populus nigra of Salicaceae (Populus trichocarpa, which may be referred to as “black cottonwood” or “cottonwood”). Examples of subspecies of Brassica rapa that are classified as members of the same species include Brassica rapa var nippo-oleifera, Brassica rapa var pekinensis, Brassica rapa var chinensis, Brassica rapa var rapa, Brassica rapa L. var. hakabura, Brassica rapa var. nipposinica, Brassica rapa var. perviridis, and Brassica rapa var chinensis (pak choi).

Examples of genes encoding a protein having the consensus sequence of Arabidopsis thaliana include the At1g54570 gene identified by the GenBank Accession Number: BT005958, the At5g41130 gene identified by the GenBank Accession Number: NM_{—}123478.3, the At5g41120 gene identified by the GenBank Accession Number: AY09638.1, and the At3g26840 gene identified by the GenBank Accession Number: AF360145.1. An example of a gene encoding a protein having the consensus sequence of barley is the FLbaf127k09 gene identified by the GenBank Accession Number: AK251457.1. Examples of genes encoding a protein having the consensus sequence of rice include the Os01g0361500 gene identified by the GenBank Accession Number: NM_{—}001049558.1, the Os01g0362100 gene identified by the GenBank Accession Number: NM_{—}001049562.1, the Os01g0361700 gene identified by the GenBank Accession Number: NM 001049559.1, and the Os09g0509500 gene identified by the GenBank Accession Number: NM_{—}001070165.1. An example of a gene encoding a protein having the consensus sequence of Brassica rapa (e.g., Brassica rapa var nippo-oleifera, Brassica rapa var pekinensis, Brassica rapa var chinensis, Brassica rapa var rapa, Brassica rapa L. var. hakabura, Brassica rapa var. nipposinica, Brassica rapa var. perviridis, and Brassica rapa var chinensis (pak choi)) is the KBrH099I08 gene identified by the GenBank Accession Number: AC189616.1. Examples of genes encoding a protein having the consensus sequence of Populus nigra include the gene that is present in the region between nucleotides 41811 and 50309, the gene that is present in the region between nucleotides 62687 and 71216, the gene that is present in the region between nucleotides 89072 and 97258, and the gene that is present in the region between nucleotides 75984 and 83278 in the complete nucleotide sequence of the clone POP002-D20, which has been registered under the GenBank Accession Number: AC213081.1.

The nucleotide sequence of the At1g54570 gene and the amino acid sequence of a protein encoded by the At1g54570 gene are shown in SEQ ID NOs: 3 and 4. The amino acid sequence of a protein encoded by the At5g41130 gene is shown in SEQ ID NO: 5. The amino acid sequence of a protein encoded by the At5g41120 gene is shown in SEQ ID NO: 6. The amino acid sequence of a protein encoded by the At3g26840 gene is shown in SEQ ID NO: 7. The amino acid sequence of a protein encoded by the FLbaf127k09 gene is shown in SEQ ID NO: 8. The amino acid sequence of a protein encoded by the Os01g0361500 gene is shown in SEQ ID NO: 9. The amino acid sequence of a protein encoded by the Os01g0362100 gene is shown in SEQ ID NO: 10. The amino acid sequence of a protein encoded by the Os01g0361700 gene is shown in SEQ ID NO: 11. The amino acid sequence of a protein encoded by the Os09g0509500 gene is shown in SEQ ID NO: 12. The amino acid sequence of a protein encoded by the KBrH099I08 gene is shown in SEQ ID NO: 13. The amino acid sequence of a protein encoded by the gene that is present in the region between nucleotides 41811 and 50309 in the complete nucleotide sequence of the clone POP002-D20, which has been registered under the GenBank Accession Number: AC213081.1, is shown in SEQ ID NO: 14. The amino acid sequence of a protein encoded by the gene that is present in the region between nucleotides 62687 and 71216 in the complete nucleotide sequence of the clone POP002-D20 is shown in SEQ ID NO: 15. The amino acid sequence of a protein encoded by the gene that is present in the region between nucleotides 89072 and 97258 in the complete nucleotide sequence of the clone POP002-D20 is shown in SEQ ID NO: 16. The amino acid sequence of a protein encoded by the gene that is present in the region between nucleotides 75984 and 83278 in the complete nucleotide sequence of the clone POP002-D20 is shown in SEQ ID NO: 17.

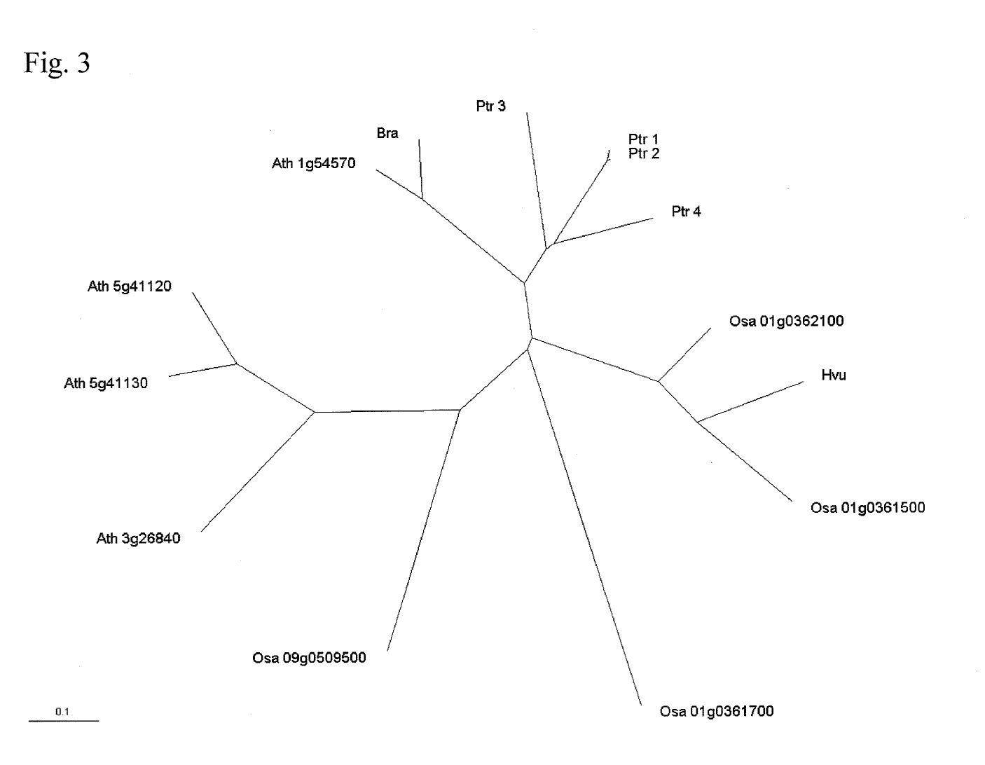

The results of alignment analysis of the amino acid sequences shown in SEQ ID NOs: 4 to 17 with the use of the CLUSTAL W (1.83) multiple sequence alignment program (available from DDBJ of the National Institute of Genetics, http://clustalw.ddbj.nig.ac.jp/top-j.html) are shown in FIG. 2 (the default scoring matrix BLOSU was used as the amino acid substitution matrix). As shown in FIG. 2, such 14 types of proteins have the above consensus sequence (the underlined region). The mutant gene of the present invention results from substitution of threonine (T) or serine (S) with another amino acid at a position indicated by an arrow in the multiple alignment shown in FIG. 2. FIG. 3 shows a molecular evolution dendrogram obtained from the results of alignment analysis of 14 types of amino acid sequences shown in FIG. 2.

As a method for substituting threonine (T) or serine (S) with another amino acid at a position indicated by an arrow in the 14 types of proteins shown in FIG. 2, a known genetic engineering technique can be adequately employed. In short, the nucleotide sequence of a wild-type gene encoding a protein into which a mutation is to be introduced is identified, and a mutation can be introduced with the use of, for example, a site-directed mutagenesis kit, so as to encode the protein after substitution. For example, the gene into which a mutation has been introduced can be incorporated into an expression vector and recovered in that state in accordance with a conventional technique. A mutation can be introduced into a gene by a known technique, such as the Kunkel or gapped duplex method, or a technique in accordance therewith. For example, a mutation is introduced with the use of mutagenesis kits utilizing site-directed mutagenesis (e.g., Mutant-K or Mutant-G (manufactured by Takara Bio Inc.)) or the LA PCR in vitro Mutagenesis Series Kit (manufactured by Takara Bio Inc.).

The mutant gene of the present invention is not limited to a gene encoding a protein comprising an amino acid sequence derived from any of the amino acid sequences as shown in SEQ ID NOs: 4 to 17 by substitution of the 12th amino acid residue from the N terminus in the aforementioned consensus sequence (i.e., threonine (T) or serine (S)) with another amino acid. The mutant gene of the present invention may comprise an amino acid sequence that results from mutation, such as deletion, substitution, or addition of a plurality of, and preferably 1 or several, amino acids of an amino acid sequence derived from any of the amino acid sequences as shown in SEQ ID NOs: 4 to 17 by substitution of the 12th amino acid residue from the N terminus in the consensus sequence as shown in SEQ ID NO: 1 (i.e., threonine (T) or serine (S)) with another amino acid. For example, 1 to 10, preferably 1 to 8, and more preferably 1 to 5 amino acids may be deleted, added, or substituted in any of the amino acid sequences as shown in SEQ ID NOs: 4 to 17.

Also, a gene encoding a protein having 70% or higher homology to any of the amino acid sequences as shown in SEQ ID NOs: 4 to 17 and having an amino acid sequence resulting from substitution of the 12th amino acid residue from the N terminus in the aforementioned consensus sequence (i.e., threonine (T) or serine (S)) with another amino acid may be used. Such homology is preferably 80% or higher, more preferably 85% or higher, further preferably 90% or higher, and most preferably 95% or higher.

Deletion, addition, or substitution of amino acids can be realized by modifying a gene encoding the above-mentioned protein via a method known in the art. A mutation can be introduced into a gene via known methods, such as the Kunkel or gapped duplex method, or methods in accordance therewith. For example, a mutation is introduced with the use of mutagenesis kits utilizing site-directed mutagenesis (e.g., Mutant-K or Mutant-G (manufactured by Takara Bio Inc.)) or the LA PCR in vitro Mutagenesis Series Kit (manufactured by Takara Bio Inc.). A mutation may be introduced into the mutant gene of the present invention via a method involving the use of chemical mutagens typified by EMS (ethyl methane sulfonate), 5-bromouracil, 2-aminopurine, hydroxylamine, N-methyl-N′-nitro-N-nitrosoguanidine, or other carcinogenic compounds, radiation processing typified by the use of x-rays, alpha rays, beta rays, gamma rays, or ion beams, or ultrasonic processing.

Further, the mutant gene of the present invention comprises DNA hybridizing under stringent conditions to DNA comprising a nucleotide sequence complementary to DNA comprising the nucleotide sequence as shown in SEQ ID NO: 3 and encoding a mutant protein having the function of regulating the oil-and-fat content in seeds. Under stringent conditions, so-called specific hybrids are formed, but non-specific hybrids are not formed. For example, hybridization is carried out at 45° C. in the presence of 6×SSC (sodium chloride/sodium citrate), followed by washing at 50° C. to 65° C. in the presence of 0.2 to 1×SSC and 0.1% SDS. Alternatively, hybridization is carried out at 65° C. to 70° C. in the presence of 1×SSC, followed by washing at 65° C. to 70° C. in the presence of 0.3×SSC.

When the nucleotide sequence of the mutant gene of the present invention is identified, the mutant gene can be obtained from a variety of plants via chemical synthesis, PCR using the cloned cDNA template, or hybridization using the DNA fragment having the nucleotide sequence of interest as a probe.

The mutant gene of the present invention described above is functionally expressed in a plant of interest by modifying the wild-type gene, so as to substitute the 12th amino acid residue from the N terminus in the aforementioned consensus sequence (i.e., threonine (T) or serine (S)) with another amino acid in the plant genome. Specifically, a plant having the mutant gene of the present invention may be produced via site-directed mutagenesis as described above. Alternatively, such plant may be produced via homologous recombination between the mutant gene prepared in advance and the wild-type gene in the genome. Further, a plant having the mutant gene of the present invention may be produced by deleting the wild-type gene in the plant genome and introducing the mutant gene in an expressible manner. Furthermore, a plant having the mutant gene of the present invention may be produced by introducing the mutant gene, so that it is overexpressed therein without deleting the wild-type gene in the plant genome.

A method of introducing the mutant gene of the present invention into a plant and a conventional method can be adequately adopted. Examples of vectors for introducing and expressing the mutant gene of the present invention in a plant cell that can be preferably used include pBI vectors, pUC vectors, and pTRA vectors. pBI and pTRA vectors can introduce a target gene into a plant with the use of Agrobacterium. pBI binary vectors or intermediate vectors are preferably used, and examples thereof include pBI121, pBI101, pBI101.2, and pBI101.3. pUC vectors can directly introduce a gene into a plant, and examples thereof include pUC18, pUC19, and pUC9. Also, plant virus vectors, such as cauliflower mosaic virus (CaMV), bean golden mosaic virus (BGMV), and tobacco mosaic virus (TMV) vectors, can be used.

The mutant gene of the present invention may not be introduced together with DNA having vector functions, but it may be directly introduced into a plant cell by the particle gun method.

The mutant gene of the present invention is preferably included in DNA that is constructed so as to exhibit the functions of such gene. In addition to a promoter, accordingly, an enhancer, a splicing signal, a poly A addition signal, a selection marker, a 5′-UTR sequence, or the like can be ligated to DNA for gene introduction, according to need. Examples of selection markers include the dihydrofolate reductase gene, the ampicillin resistance gene, the neomycin resistance gene, the hygromycin resistance gene, and the bialaphos resistance gene.

As the “promoter,” DNA may not be derived from a plant, provided that such DNA can function in a plant cell and induce expression in a given tissue or at a given growth phase of the plant. Specific examples include 35S promoters of cauliflower mosaic virus (CaMV), promoters of nopaline synthase genes (Pnos), maize or rice ubiquitin promoters, maize or rice actin promoters, and tobacco PR protein promoters.

Any sequence may be used as the “terminator,” provided that such sequence can terminate the transcription of the gene transcribed by the promoter. Specific examples include terminators of noparine synthase genes (Tnos) and cauliflower mosaic virus poly A terminators.

An “enhancer” is used for improving the expression efficiency of a target gene. For example, an enhancer region including a sequence located upstream in the CaMV35S promoter is preferable.

A transgenic plant can be prepared in accordance with a conventional technique using an expression vector having the mutant gene of the present invention. A transgenic plant can be obtained by introducing such expression vector into a host cell, so that the introduced mutant gene can be expressed therein. If the mutant gene of the present invention is included in a DNA fragment, which is constructed so as to exhibit the functions of the mutant gene, a transgenic plant can be prepared by the particle gun method while lacking DNA having vector functions. A transgenic plant can be prepared in the following manner. Targets of transformation are plant tissues (including the epidermis, the phloem, the parenchyma, the xylem, the vascular bundle, and the plant organs (e.g., leaves, petals, stems, radicals, and seeds)) or plant cells.

Examples of plants having the mutant gene of the present invention include, but are not limited to, dicotyledonous plants and monocotyledonous plants, such as plants of Brassicaceae, Gramineae, Solanaceae, Leguminosae, and Salicaceae (see below).

Examples of Brassicaceae plants include Arabidopsis thaliana, oilseed rape (Brassica rapa and Brassica napus), cabbage (Brassica oleracea var. capitata), rapeseed (Brassica rapa and Brassica napus), field mustard (Brassica rapa and Brassica napus), Brassica pekinensis (Brassica rapa var. pekinensis), bok choy (Brassica rapa var. chinensis), turnip (Brassica rapa var. rapa), Brassica rapa var. hakabura, Brassica rapa var. lancinifolia, Brassica rapa var. peruviridis, pak choi (Brassica rapa var. chinensis), Japanese radish (Brassica Raphanus sativus), and horseradish (Wasabia japonica).

Examples of Solanaceae plants include tobacco plants (Nicotiana tabacum), eggplants (Solanum melongena), potatoes (Solaneum tuberosum), tomatoes (Lycopersicon lycopersicum), capsicum (Capsicum annuum), and Petunia.

Examples of Leguminosae plants include soybeans (Glycine max), peas (Pisum sativum), horse beans (Vicia faba), Wisteria floribunda, peanuts (Arachis. hypogaea), bird's-foot trefoils (Lotus corniculatus var. japonicus), bush beans (Phaseolus vulgaris), azuki beans (Vigna angularis), and Acacia.

Examples of Compositae plants include Chrysanthemums (Chrysanthemum morifolium) and sunflowers (Helianthus annuus).

Examples of Arecaceae plants include Elaeis guineensis (or Elaeis oleifera), Cocos nucifera, Phoenix dactylifera, and Copernicia.

Examples of Anacardiaceae plants include Rhus succedanea, Anacardium occidentale, Toxicodendron vernicifluum, Mangifera indica, and pistachio (Pistacia vera).

Examples of Cucurbitaceae plants include pumpkins (Cucurbita maxima, Cucurbita moschata, or Cucurbita pepo), cucumbers (Cucumis sativus), Trichosanthes cucumeroides, and gourds (Lagenaria siceraria var. gourda).

Examples of Rosaceae plants include almonds (Amygdalus communis), roses (Rosa), strawberries (Fragaria), cherry trees (Prunus), and apples (Malus pumila var. domestica).

Examples of Caryophyllaceae plants include carnations (Dianthus caryophyllus).

Examples of Salicaceae plants include Populus nigra (Populus trichocarpa, Populus nigra, or Populus tremula).

Examples of Gramineae plants include maize (Zea mays), rice (Oryza sativa), barley (Hordeum vulgare), wheat (Triticum aestivum), bamboo (Phyllostachys), and sugarcane (Saccharum officinarum).

Examples of Liliaceae plants include tulips (Tulipa) and lilies (Lilium).

Examples of methods for introducing an expression vector or a DNA fragment having the mutant gene of the present invention into a plant include the Agrobacterium method, the PEG-calcium phosphate method, the electroporation method, the liposome method, the particle gun method (the bombardment method), and the microinjection method. When the Agrobacterium method is employed, for example, protoplasts or tissue sections are used. When protoplasts are used, protoplasts may be subjected to coculture with Agrobacterium having Ti plasmids or may be fused to Agrobacterium spheroplasts (i.e., the spheroplast method). When tissue sections are used, tissue sections may be infected with an aseptically-cultured leaf disc of a target plant via a leaf disk (the leaf disc method), tissue sections may be infected with calluses (undifferentiated cultured cells), or tissue sections may directly infiltrate flower tissue. When transforming monocotyledonous plants by the Agrobacterium method, acetosyringone can be used to enhance the efficiency of transformation.

Whether or not the mutant gene has been incorporated into a plant can be confirmed via PCR, Southern hybridization, Northern hybridization, or other techniques. For example, DNA is prepared from a transgenic plant, DNA-specific primers are designed, and PCR is carried out. After PCR is performed, the amplification product may be subjected to agarose gel electrophoresis, polyacrylamide gel electrophoresis, or capillary electrophoresis, the resultant is stained with ethidium bromide, SYBR Green, or the like, and the amplification product is detected as a band. Thus, transformation can be confirmed. Alternatively, PCR may be carried out with the use of primers that have been labeled with fluorescent dyes or the like in advance, and the amplification product can be detected. Further, the amplification product may be bound to a solid phase, such as a microplate, and the amplification product may be detected via fluorescent, enzyme, or other reactions.

As a result of transformation, the resulting tumor tissues, shoots, capillary roots, seeds, or the like can be used for cell culture, tissue culture, or organ culture in that state. Alternatively, conventional plant tissue culture techniques may be employed to reproduce plants therefrom via administration of plant hormones (e.g., auxin, cytokinine, gibberellin, abscisic acid, ethylene, or brassinoride) at adequate concentrations. In general, plants are reproduced from the cultured cell by differentiating the radicals on a medium containing a mixture of adequate types of auxin and cytokinine, transferring the resultants to a medium containing a larger quantity of cytokinine to differentiate shoots, and transferring the resultants to hormone-free soils.

Thus, a transgenic plant into which the mutant gene has been introduced exhibits a characteristic phenotype. That is, the total amount of oil-and-fat in a given tissue of the transgenic plant is increased in comparison with that of a wild-type plant into which the mutant gene has not been introduced; however, the oil-and-fat content (%) in the given tissue is decreased in comparison with that of a wild-type plant. In this case, a given tissue may be, for example, a leaf, cotyledon, radical, radical hair, trichome, flower, seed, fruit, scape, radical tuber, tuber, or bulb. A cotyledon, seed, or fruit is preferable, and a seed is more preferable. The oil-and-fat component in seeds obtained from a plant can be quantitatively detected via a conventional technique. The oil-and-fat component in a given tissue can be measured by a method involving the use of a pulsed NMR apparatus, for example.

The mutant gene of the present invention was identified by visualizing, with fluorescence, oil bodies contained in the cotyledons sampled from the transgenic Arabidopsis thaliana plant into which the oleosin-GFP fusion gene had been introduced. In the present invention, however, oil-body-specific proteins are not limited to oleosins, and a wide variety of proteins having oil body specificity can be used. Examples of oil-body-specific proteins include oleosins, steroleosins, and caleosins. In the present invention, any protein may be used as a fluorescent protein that allows visualization of oil bodies in cotyledons, provided that such protein has fluorescence ability. Examples of proteins that can be used include GFP (green fluorescent protein), YFP (yellow fluorescent protein), RFP (red fluorescent protein), OFP (orange fluorescent protein), and BFP (blue fluorescent protein). Such proteins are not limited to fluorescent proteins, as long as the protein allows visualization of oil bodies in cotyledons. A wide variety of proteins that can detect oil bodies with the aid of visible light can be used. A specific example of a protein that can detect oil bodies with the aid of visible light is a luminescent protein, such as luciferase.

Hereafter, the present invention is described in greater detail with reference to the examples, although the technical scope of the present invention is not limited to the following examples.

### Example 1

In this example, Arabidopsis thaliana plants that are extensively used as model plants were transformed so as to express an oleosin-GFP fusion gene, and transgenic plants in which oil bodies can be observed with the aid of fluorescence were prepared. Thereafter, the resulting transgenic plants were subjected to mutation, and mutants exhibiting varied oil-and-fat amounts in seeds were identified using changes in oil body properties as an indicator. Thereafter, the causal genes in the identified mutants were specified, the genes having the function of varying the oil-and-fat amount in seeds were identified, and a mutation of amino acid substitution that varies the oil-and-fat amount in plants was identified. Hereafter, the concrete process of experiment and the results of experiment are described in detail.

### Material and Method

### <Plant Material>

The Arabidopsis thaliana ecotype Columbia was used. In accordance with a conventional technique, plants were subjected to seed sterilization and germination in sterile agar medium (½ Murashige and Skoog medium, 0.8% agar) for 7 days at 22° C. under light. Thereafter, plants were planted in a pot containing vermiculite and pearlite (1:1) and grown at 22° C. for 16 hours under light and for 8 hours in the dark.

### <Preparation of Oleosin-GFP Gene>

RNA was isolated from the sheath of Arabidopsis thaliana using an RNeasy plant mini kit (Quiagen) and subjected to reverse transcription using the SuperScript III first strand synthesis system for RT-PCR (Invitrogen). PCR was carried out using the obtained cDNA, primer 1 (^{5′}AAAAAGCAGGCTCAATGGCGGATACAGCTAGAGGA^{3′}: SEQ ID NO: 18), and primer 2 (^{5′}CTCGCCCTTGCTCACCATAGTAGTGTGCTGGCCACC^{3′}: SEQ ID NO: 19), and DNA fragment A having part of the attB1 sequence and part of the GFP gene at both ends of oleosin S3 cDNA was amplified. Separately, PCR was carried out using cDNA encoding the green fluorescent protein (GFP) from Aequorea victoria, primer 3 (^{5′}GGTGGCCAGCACACTACTATGGTGAGCAAGGGCGAG^{3′}: SEQ ID NO: 20), and primer 4 (^{5′}AGAAAGCTGGGTCTTACTTGTACAGCTCGTCCAT^{3′}: SEQ ID NO: 21), and DNA fragment B having part of oleosin S3 cDNA and part of the attB2 sequence added to both ends of GFP cDNA was amplified. Subsequently, DNA fragment A, DNA fragment B, primer 5 (^{5′}GGGG ACA AGT TTG TAC AAA AAA GCA GGC T^{3′}: SEQ ID NO: 22), and primer 6 (^{5′}GGGG AC CAC TTT GTA CAA GAA AGC TGG G^{3′}: SEQ ID NO: 23) were mixed, and PCR was further carried out to prepare an oleosin-GFP fusion gene having the attB1 sequence and the attB2 sequence at both ends. The nucleotide sequence of the oleosin-GFP fusion gene and the amino acid sequence of the gene product are shown in SEQ ID NOs: 24 and 25.

The obtained fusion gene was cloned into a Ti vector having the attR1 and attR2 sequences downstream of the CaMV 35S promoter and having a kanamycin-resistant marker with the aid of the pDONR221 vector in accordance with the protocol of the Gateway system (Invitrogen). The obtained plasmid was introduced into Agrobacterium (Agrobacterium tumefacience C58C1 rifR) via electroporation, and the resultant was designated as Ti-OleG.

<Transformation into Arabidopsis Thaliana>

The oleosin-GFP fusion gene was introduced into the genome of Arabidopsis thaliana by the Agrobacterium method. At the outset, Ti-OleG was allowed to grow in YEB medium (5 g/l polypeptone, 5 g/l beef extract, 1 g/l yeast extract, 5 g/l sucrose, 0.5 g/l MgSO₄) until the absorbance at 600 nm (A600) reached 0.8 to 1.0 at 28° C., and it was then harvested via centrifugation. The resulting cells were suspended in an infiltration solution (10 mM MgCl₂, 5% sucrose, 0.05% Silwet L-77) so as to adjust A600 at 0.8. After scapes of flowering Arabidopsis thaliana were soaked in the suspension for 1 minute, fructified seeds were collected. The collected seeds were sterilized and then sowed in sterile agar medium containing 25 mgm/l kanamycin, and the transgenic Arabidopsis thaliana plant into the genome of which the oleosin-GFP fusion gene had been inserted was isolated using kanamycin resistance as an indicator. Seeds were obtained from the resulting transgenic Arabidopsis thaliana plant, and progenies of the transgenic plants, which were homozygous for the kanamycin resistance marker, were selected and designated as OleG.

### <Transgenic Plant Mutagen Treatment>

OleG seeds were treated with a 0.2% ethyl methane sulfonate solution for 16 hours and then sowed in a pot containing vermiculite and pearlite (1:1). The seeds were grown at 22° C. under light for 16 hours and in the dark for 8 hours, and the progeny seeds were collected and designated as M2 seeds.

### <GFP Fluorescence Observation>

Mutants were screened for using a fluorescence stereomicroscope (Carl Zeiss SteREO Lumar V 12). The OleG and M2 seeds were vertically positioned on sterile agar medium and germinated in the dark for 6 days. The GFP fluorescence of the oleosin-GFP fusion protein in cells of etiolated cotyledons, embryonic axes, and radicals was observed under a fluorescence stereomicroscope (Carl Zeiss). Seeds having GFP fluorescence intensities and distributions different from those of OleG were identified as mutants.

In order to compare GFP fluorescence of the oleosin-GFP fusion protein of the OleG and mutant seeds, a confocal laser microscope (Carl Zeiss LSM 510) was used. Etiolated cotyledons, embryonic axes, and radicals were cut from the OleG and mutant seeds, which had been germinated in the dark for 6 days, and they were then mounted on a glass slide. GFP fluorescent images of the cells were photographed under the same conditions, the frequency distribution of fluorescence intensity relative to pixels within the same area was calculated using image analyzing software included with the microscope, and the fluorescence sum (i.e., a=sum; fluorescence intensity×pixel number) was determined.

### <Electrophoresis and Immunoblot Analysis of Seed Protein>

20 seed grains were pulverized in 40 μl of SDS sample buffer, and the supernatant resulting from centrifugation was designated as a seed protein sample. In accordance with a conventional technique, 15 μl of the sample was subjected to SDS polyacrylamide electrophoresis. The electrophoresed gel was stained with a solution of 0.2% Coomassie brilliant blue R-250 (containing 25% methanol and 10% acetic acid).

Immunoblot analysis was carried out by subjecting 5 μl of the sample to SDS polyacrylamide gel electrophoresis and transferring the proteins in the gel onto a nitrocellulose membrane via semi-dry blotting. Detection of the proteins that had been transferred onto the nitrocellulose membrane with the use of an anti-protein antibody was carried out in accordance with the protocols of GE Healthcare Bio-Sciences using ECL Western blotting detection reagents. In this case, 1/5000-fold diluted antibodies were used as the primary antibodies (the anti-oleosin antibodies or the anti-GFP antibodies) and the secondary antibodies. Luminescence was detected using a luminescent image analyzer (LAS-1000 plus, Fujifilm Corporation).

### <Electron Microscopic Seed Cell Observation>

Half seeds were immobilized using a fixative (4% paraformaldehyde, 1% glutaraldehyde, 10% DMSO, 0.05M cacodylate buffer (pH 7.4)). The immobilized sample was embedded in epon 812 resin and ultrathin sections were prepared using a Leica Microtome Ultracut UCT. The ultrathin sections were subjected to electron staining with 4% uranium acetate and 0.4% lead citrate and then observed under an electron microscope (H-7600, Hitachi, Ltd.).

### <Measurement of Oil-and-Fat Amount in Seed>

While performing neutralization of static electricity, the seeds were weighed with an accurate electron balance using drug packing paper, and 10 to 12 mg of seeds were fractionated. The seeds were introduced into a test tube for pulsed NMR, and the oil-and-fat content in seeds (% by weight) was determined using a MARAN-23 pulsed NMR (Resonance Instruments) based on the ¹H-pulsed NMR relaxation times. The detailed measurement procedure described in the instructions for pulsed-NMR measurements was employed.

### <Analysis of Fatty Acid Composition in Seed Oil-and-Fat>

About 1 mg to 5 mg of seed samples were fractionated and introduced into a 1.5-ml micro test tube. A tungsten carbide bead (φ=3 mm) was added to the micro test tube, and 450 μl of methanol, 50 μl of a solution of butyl hydroxyl toluene mixed with a methanol solvent at 0.2% (w/v), and 10 μl of 0.2% C15:0 fatty acid as an internal standard material were further added. The micro test tube to which such various reagents and the sample had been added was subjected to oscillation at a frequency of 1/20 s using a Retsch Mixer Mill Type MM301 for 1 minute, and the seeds were pulverized. The sample was transferred to a 10-ml test tube with a screw cap. Further, the inside of the micro test tube was washed twice with 250 μl of methanol, and the methanol wash solution was added to the test tube to adjust the amount of the sample solution to about 1 ml. A 10% hydrochloric acid/methanol solution (1 ml) was added thereto, the mixture was treated at 80° C. for 1 hour, 1.5 ml of n-hexane was added thereto, the mixture was agitated using a vortex mixer, and the n-hexane layer was transferred to a 10-ml spit tube. Further, the inside of the test tube used for methanolysis was washed with 1 ml of n-hexane, and the n-hexane-washed layer was added to the spit tube. The resulting n-hexane solvent solution was subjected to nitrogen gas purging at 40° C., and fatty acid methyl ester was dried. The dried fatty acid methyl ester was dissolved in 500 μl of n-hexane, and various fatty acid methyl esters were separated and quantified via GC-FID. Quantification was carried out with reference to the area of the internal standard (C15:0 fatty acid).

### <Identification of Mutant Gene>

After the mutagen treatment, mutants having a Columbia ecotype background were crossed with wild-type Arabidopsis thaliana plants (i.e., the ecotype Landsberg erecta) in order to perform high-precision mapping of causal genes for mutants exhibiting changed oil-and-fat amounts in seeds, and 310 lineages of the F2 generation that were homozygous for the mutant gene were obtained. In accordance with a conventional technique, the genetic backgrounds of these 310 lineages of chromosomes (620 chromosomes in total) were scored using many molecular markers. The gene that was deduced to be the causal gene based on the genome DNAs of the OleG and mutant seeds was amplified via PCR, and the nucleotide sequence thereof was determined using a 3130xl Genetic Analyzer (Applied Biosystems).

### Results and Discussion

### <Establishment of Method for Screening for Mutant That is Deficient in Oil Body Formation>

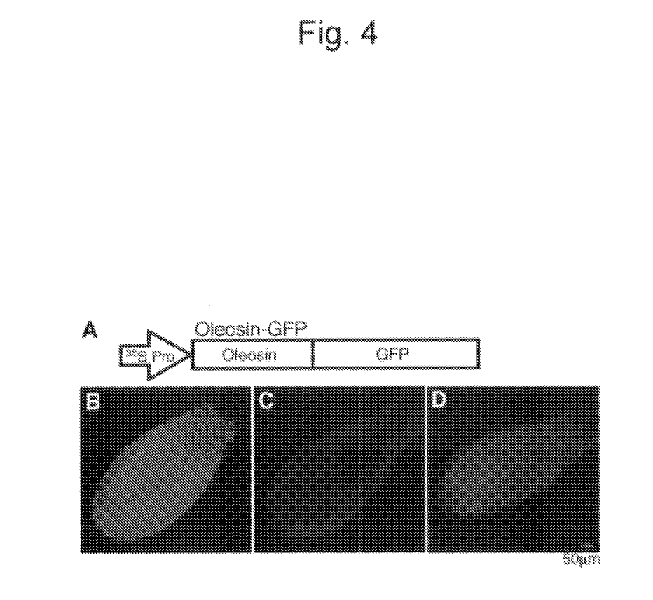

The fusion gene (oleosin-GFP) encoding the fusion protein of oleosin and green fluorescent protein (GFP) was prepared and then ligated to a site downstream of DNA of the 35S promoter of the cauliflower mosaic virus (FIG. 4A). The DNA construct was introduced into genome DNA of Arabidopsis thaliana by the Agrobacterium method to prepare a transgenic Arabidopsis thaliana plant, and the resultant was designated as oleG. The results of observation of oleG cotyledons, which had been germinated in the dark for 6 days, using a fluorescent microscope are shown in FIG. 4B. As is apparent from FIG. 4B, an oil body membrane is labeled with GFP fluorescence, and aggregates of many small oil bodies are present. Further, the presence of oil bodies in germs and green cotyledons, true leaves, and petals, which had been germinated under light, was observed, in addition to cotyledons, which had been germinated in the dark.

In order to identify the gene associated with the mechanism for accumulating plant oil-and-fats in oil bodies, the oleG seeds were subjected to mutation with ethyl methane sulfonate to obtain progeny M2 seeds. The M2 plants, which had been germinated in the dark for 6 days, were observed under a fluorescent microscope, and mutant A (FIG. 4C) and mutant B (FIG. 4D) exhibiting fluorescent intensity different from that of oleG were obtained. The GFP fluorescent intensity in the germinated cotyledons of such mutants was lower than that of oleG.

### <Correlation Between Sum of GFP Fluorescence and Oil-and-Fat Content in Seed>

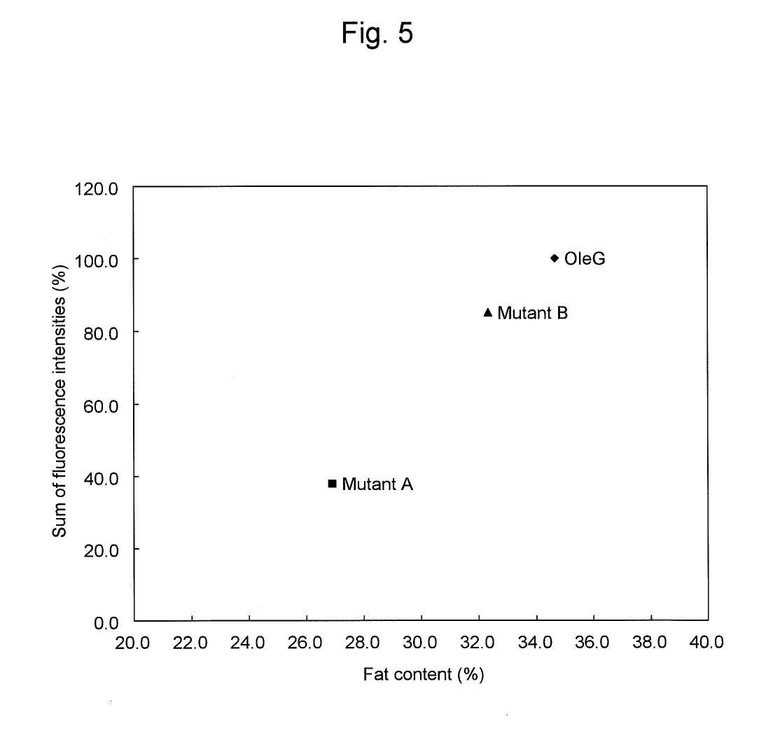

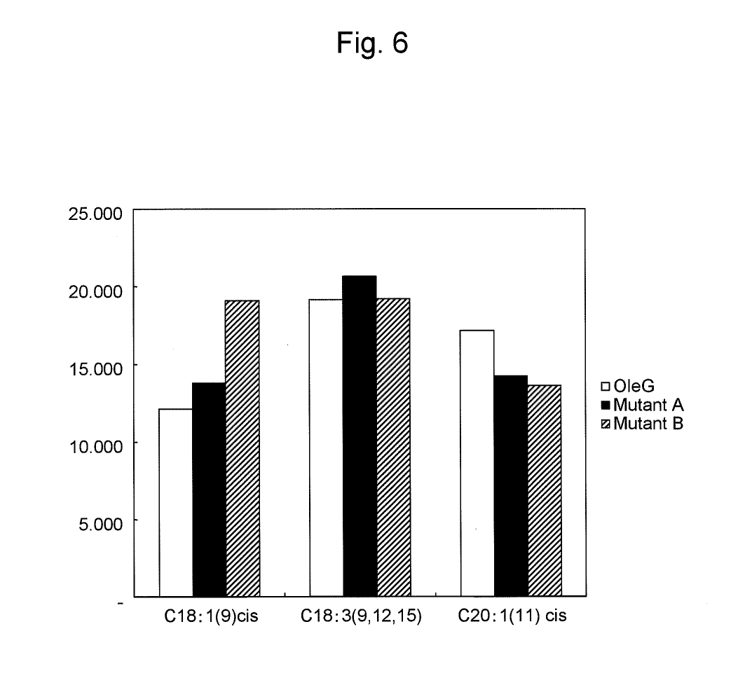

Confocal laser microscope photographs of GFP fluorescence of cotyledons of OleG, mutant A, and mutant B, which had been germinated in the dark for 6 days, under the same conditions in the same area with the same pixel number were obtained, and the sum of GFP fluorescence (i.e., a=sum; fluorescence intensity×pixel number) was determined based on the frequency distribution of the pixel number relative to the GFP fluorescence intensity of each photograph. When the sum of OleG fluorescence was designated as 100%, the fluorescence sum of mutant A was 37.9%, and that of mutant B was 85.1%. Separately, the oil-and-fat contents in seeds of OleG, mutant A, and mutant B were measured and found to be 34.66%±0.43%, 26.91%±0.34%, and 32.34%±0.49%, respectively. A positive correlation was observed between the sum of GFP fluorescence and the oil-and-fat content in seeds (FIG. 5). Also, the fatty acid compositions in lipids accumulated in seeds of such mutants were analyzed (FIG. 6). As a result, the C20:1(11)cis content in mutant A and mutant B were found to be lower than that in OleG. In this description, notation regarding a fatty acid is provided in the following manner; C(the number of carbon atoms:“the number of unsaturated carbon bonds”“(the carbon number counted from the carboxyl group side where an unsaturated bond exists)”“the cis/trans geometric isomerization of unsaturated bonds”. When a cis/trans geometric isomerism is not known, notation regarding such isomerism is not provided. For example, stearic acid is represented as C18:0 and general oleic acid is represented as C:18:1(9)cis. In mutant A, an increase was observed in C18:3 (9, 12, 15), and an increase was observed in C18:1(9)cis in mutant B. Thus, measurement of the sum of GFP fluorescence of the oleosin-GFP fusion protein was found to enable identification of a mutant having an abnormality in the mechanism for accumulating plant oil-and-fats alive.

In FIG. 6, bar charts representing fatty acids represent OleG, mutant A, and mutant B in that order from the left.

### <Analysis of Seed Protein>

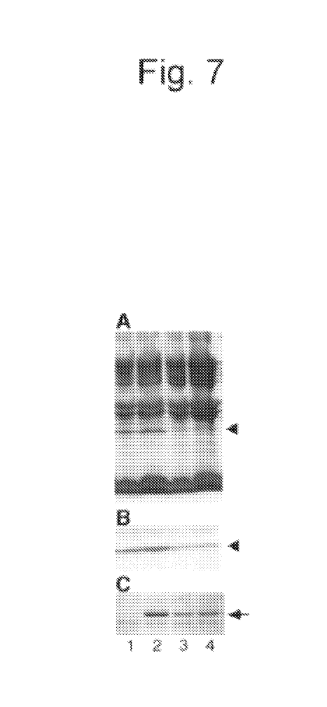

FIG. 7 shows the results of analysis of proteins contained in seeds of a wild-type Arabidopsis thaliana plant, OleG, mutant A, and mutant B. The results of Coomassie brilliant blue staining (FIG. 7A), immunoblot analysis using the anti-oleosin antibody (FIG. 7B), and immunoblot analysis using the anti-GFP antibody (FIG. 7C) demonstrate that the amounts of endogenous oleosins and of oleosin-GFP decreased in seeds of mutant A and mutant B, in comparison with those of OleG. In FIGS. 7A to 7C, lane 1 represents a sample derived from a wild-type Arabidopsis thaliana plant, lane 2 represents a sample derived from OleG, lane 3 represents a sample derived from mutant A, and lane 4 represents a sample derived from mutant B.

### <Electron Microscopic Observation of Oil Bodies>

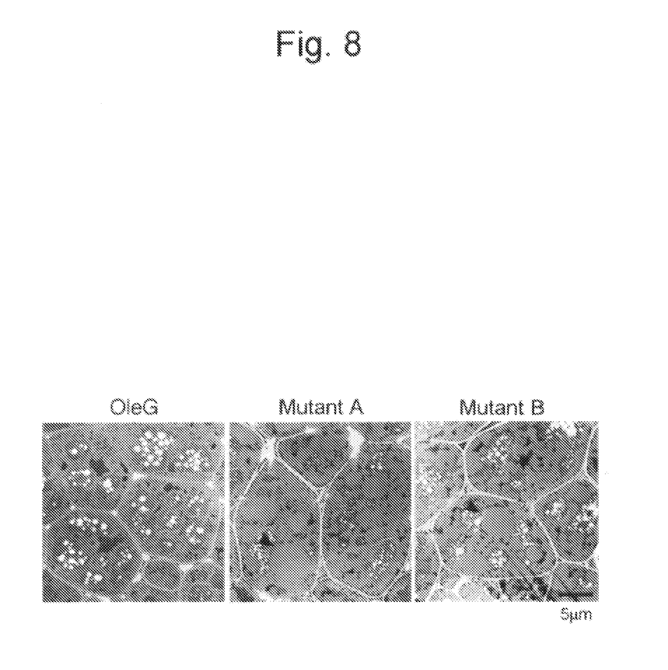

FIG. 8 shows the results of observation of OleG, mutant A, and mutant B seed cells under an electron miscroscope. OleG seed cells are filled with small oil bodies, as in the case of wild-type Arabidopsis thaliana plants. In contrast, gaps that are deduced to be cytosols are observed between oil bodies, and each oil body is round in mutant A and mutant B. This is considered to result from a decreased amount of seed reserved lipid. Also, several oil bodies in seeds are gigantic. This is considered to result from a decreased amount of oleosin in the oil body membrane.

### <Identification of Causal Gene>

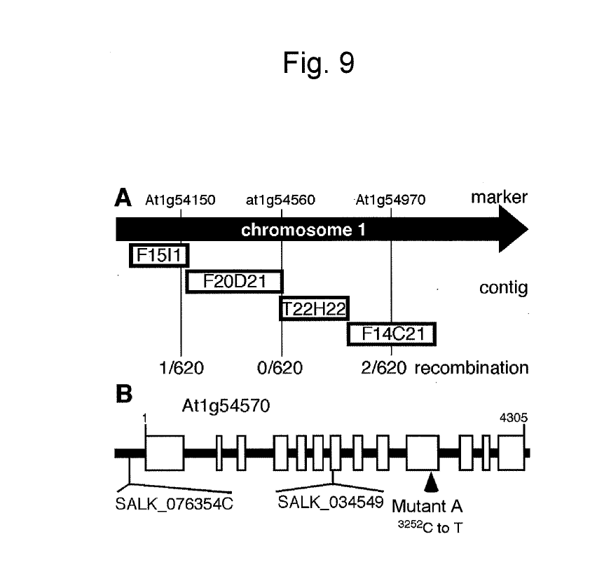

Mutant A having a Columbia ecotype background was crossed with an wild-type Arabidopsis thaliana plant having a Landsberg erecta ecotype background, and high-precision mapping of causal genes was carried out. The results of scoring of the genetic backgrounds of 620 chromosomes with the use of molecular markers are shown in FIG. 9A. The number of Landsberg erecta-derived chromosomes indicates that recombination took place between the locus of the causal gene of mutant A and a molecular marker. As shown in FIG. 9A, high-precision mapping demonstrates that the locus of the causal gene of mutant A is located in a position very close to the first chromosome At1g54560, which is between At1g54150 and At1g54970. The presence of 87 genes including the At1g54570 gene, whose functions are unidentified (FIG. 9B), is suggested in this region.

At1g54570 encodes a polypeptide including a portion that is proposed as the diacylglycerol acyltransferase (DAGAT) domain. The DAGAT domain is an amino acid sequence that is considered to be commonly present in an enzyme associated with triacylglycerol synthesis (i.e., diacylglycerol acyltransferase). Since this is a deduction based on nucleotide sequence information, polypeptides having the DAGAT domain do not always have DAGAT activity. Also, research regarding DAGAT has been conducted mainly with the use of budding yeast, and no case is known in which a given polypeptide in plant seeds has been identified as DAGAT.

The nucleotide sequence of the At1g54570 gene region located in genome DNA of mutant A was analyzed. As a result, a point mutation of C3252T was observed in the nucleotide sequence of At1g54570 (the notation “C3252T” indicates substitution of C with T at nucleotide 3252, when A of the initiation codon ^{5′}ATG^{3′} of cDNA relative to mRNA is designated as 1). This point mutation is present in exon 10, and it induces missense mutation of T5081 in the DAGAT domain of the amino acid sequence of the At1g54570 gene product (the notation “T508I” indicates a substitution of T(Thr) at the 508th amino acid residue from the N-terminus of a polypeptide with I(Ile)). Thus, it was concluded that the causal gene of mutant A is At1g54570, and the mutation of T508I in the At1g54570 gene product is an amino acid substitution that varies the oil-and-fat amount in a plant.

### INDUSTRIAL APPLICABILITY

The mutant gene of the present invention can regulate the oil-and-fat content or oil-and-fat composition in seeds. According to the method for regulating the oil-and-fat content in seeds of the present invention, a plant with the regulated oil-and-fat content or oil-and-fat composition can be provided.

All publications, patents, and patent applications cited herein are incorporated herein by reference in their entirety.

1. A mutant gene encoding a mutant protein having a consensus sequence derived from the amino acid sequence as shown in SEQ ID NO: 1 by substitution of the 12th amino acid residue from the N terminus with another amino acid and having the function of regulating the oil-and-fat content in plant seeds.

2. The mutant gene according to claim 1, wherein the other amino acid is an amino acid selected from the group consisting of isoleucine, valine, leucine, and methionine.

3. The mutant gene according to claim 1, wherein the other amino acid is isoleucine.

4. The mutant gene according to claim 1, wherein the mutant protein has an amino acid sequence derived from an amino acid sequence as shown in any one of SEQ ID NOs: 4 to 17 comprising a substitution of the 12th amino acid residue from the N terminus in the consensus sequence with another amino acid.

5. The mutant gene according to claim 1, which is a mutant of a plant-derived gene.

6. The mutant gene according to claim 1, which is a mutant of a gene derived from a plant belonging to a family selected from the group consisting of Gramineae, Brassicaceae, and Salicaceae.

7. The mutant gene according to claim 1, which is a mutant of a gene derived from a plant belonging to a species selected from the group consisting of barley, rice, Arabidopsis thaliana, Brassica rapa, and Populus nigra.

8. A mutant protein encoded by the mutant gene according to claim 1.

9. A transgenic cell into which the mutant gene according to claim 1 is introduced.

10. A transgenic plant into which the mutant gene according to claim 1 is introduced.

11. A method for regulating the oil-and-fat content in seeds comprising introducing a mutation into a gene encoding a protein having a consensus sequence comprising the amino acid sequence as shown in SEQ ID NO: 1, wherein the mutation id a substitution of the 12th amino acid residue from the N terminus of the amino acid sequence as shown in SEQ ID NO: 1 with another amino acid.

12. The method for regulating the oil-and-fat content in seeds according to claim 11, wherein the other amino acid is an amino acid selected from the group consisting of isoleucine, valine, leucine, and methionine.

13. The method for regulating the oil-and-fat content in seeds according to claim 11, wherein the other amino acid is isoleucine.

14. The method for regulating the oil-and-fat content in seeds according to claim 11, wherein a mutant protein resulting from substitution of the 12th amino acid residue from the N terminus with another amino acid in the consensus sequence included in an amino acid sequence as shown in any one of SEQ ID NOs: 4 to 17 is expressed.

15. The method for regulating the oil-and-fat content in seeds according to claim 11, wherein a gene derived from a plant belonging to a family selected from the group consisting of Gramineae, Brassicaceae, and Salicaceae is varied.

16. The method for regulating the oil-and-fat content in seeds according to claim 11, wherein a gene derived from a plant belonging to a species selected from the group consisting of barley, rice, Arabidopsis thaliana, Brassica rapa, and Populus nigra is varied.


SEQUENCE LISTING (25 sequences)

SEQ ID NO: 1 - protein, 80 aa, Artificial Sequence
  Synthetic consensus sequence including a DAGAT domain
XXXXXXGXXXXXXXXXXXLXXXXXXVXLXPGGXREXLHXXXEXXXLXWPXXXEFVRXAXXFXXXXXPFGXVGEDDXXXXX

SEQ ID NO: 2 - protein, 90 aa, Artificial Sequence
  Synthetic consensus sequence including a DAGAT domain
XXXXXXGXXXXXXXXXXXLXXXXXXVXLXPGGXREXLHXXXEXXXLXWPXXXEFVRXAXXFXXXXXPFGXVGEDDXXXXXXXXXXXXXXP

SEQ ID NO: 3 - DNA, 2115 bp, Arabidopsis thaliana
  CDS (1)..(2115)
atggcaacatgttcttcaagcttattggtgttaccaaatttgagattgagttctaaccaaagacgcaatttcaaggtaagagctcaaatctctggtgaaaacaagaaggcgacatcattggaaccagtgaacaacaacggttcggtctctttgtctactactgttcaaaaccaaaaaggggcaaatgaagtgaatgggaaagggaaatccaagagaaagatagtgagtgatgaaattgagttgttatgggatgatgggtatggctccaaatctgttaaagattactttgctgcggctaaagagattttgaaggctgatggtggtcctccacggtggtttagtcccgttgattgtggtcgacctgttgaagatgctcctacgttactcttcttacctgggatggatggcacagggatgggccttgttccacatcacaaagctcttggcaaggcttttcatgtctcgtgcctgcatattcctgtacttgatcgaactccatttgaagggcttctgaaagtagtcgaagatgttttgaggcaggagcaggctacacgtcctaataagccgatatatctcgtgggcgattcctttggaggatgcctggctcttgctgttgctgctagaaaccgttcgttagacttggtgttgatactagtcaacccagctacatcatttgataggtcaccgctacagccgttgctgcctatattagagatggtgccagaggagcttcattttactgttccatatgctctaagctttattatgggtgatccgataaagatggcgacacttggtatcgacaaccaacttccaacaggagtaaaaattgaaaagttgagacaaaggcttactaaaacaatgctcccgcttctctctgagctcggtggaattattccacgggaaaccttactttggaagcttaagttactgagatctggctgtgcttatgccaattcccgcatccatgctgttcaagctgaagtcttggtccttgctagtggaaaggacatgatgcttcctagccaagaagaagcaaagcggcttcacgggttacttaaaaactgttcagttcgatgcttcaaggacaatgggcatacccttttattggaagatagcatcagtttgctcacagttataaaaggtacaggcaagtaccggcggtcctggagatatgatttggtttctgatttcttgcctccaagtaagggagaacttgcttacgcccttgatgaagtgcttggattcttgcgaaatgctgttggttctgtgtttttttcaactatggaagacgggaaaatagtgaagggacttgctggtgttccagataaaggtccggtgctacttgttggttaccacatgttgatgggtcttgaacttggtccaatgtccgaggcgtttataaaagagaagaacattctctttcgtggaatggcccatccggttctgtattctgataatgatccagctaaggcgtttgattatggtgactggattaaagtatttggtgcctatcctgtcaccgcaaccaatctatttaagctgttagattctaaatctcatgtccttctcttccctggtggtgcacgagaggctctccataaccggggtgaacaatacaaactaatttggcctgaacagcaagagtttgtaagaatggcagcacggtttggtgcaacaatagtgccgtttggaacagttggtgaagacgacatagctgaattggttcttgattacaacgacctgatgaagatccctattcttaatgactatatcacggaagttactcgcgataccaaacaatttaaattgagggaagagtcagagggagaggtggcaaaccagccactctacctaccaggactcataccgaaagtcccgggtaggttctattacctgtttgggaaaccgatagagacaaagggaagaccagagcttgtaaaagacaaagaagaagcaaatcaagtttacttggaggtaaaggcggaggtagagaatagtatagcctacctgttgaagaaacgagaggaagatccatacagaagtgtcttagacagattaaactacagtttgacgcacactaccgcaactcatgttccatctttcgagccatga

SEQ ID NO: 4 - protein, 704 aa, Arabidopsis thaliana
MATCSSSLLVLPNLRLSSNQRRNFKVRAQISGENKKATSLEPVNNNGSVSLSTTVQNQKGANEVNGKGKSKRKIVSDEIELLWDDGYGSKSVKDYFAAAKEILKADGGPPRWFSPVDCGRPVEDAPTLLFLPGMDGTGMGLVPHHKALGKAFHVSCLHIPVLDRTPFEGLLKVVEDVLRQEQATRPNKPIYLVGDSFGGCLALAVAARNRSLDLVLILVNPATSFDRSPLQPLLPILEMVPEELHFTVPYALSFIMGDPIKMATLGIDNQLPTGVKIEKLRQRLTKTMLPLLSELGGIIPRETLLWKLKLLRSGCAYANSRIHAVQAEVLVLASGKDMMLPSQEEAKRLHGLLKNCSVRCFKDNGHTLLLEDSISLLTVIKGTGKYRRSWRYDLVSDFLPPSKGELAYALDEVLGFLRNAVGSVFFSTMEDGKIVKGLAGVPDKGPVLLVGYHMLMGLELGPMSEAFIKEKNILFRGMAHPVLYSDNDPAKAFDYGDWIKVFGAYPVTATNLFKLLDSKSHVLLFPGGAREALHNRGEQYKLIWPEQQEFVRMAARFGATIVPFGTVGEDDIAELVLDYNDLMKIPILNDYITEVTRDTKQFKLREESEGEVANQPLYLPGLIPKVPGRFYYLFGKPIETKGRPELVKDKEEANQVYLEVKAEVENSIAYLLKKREEDPYRSVLDRLNYSLTHTTATHVPSFEP

SEQ ID NO: 5 - protein, 688 aa, Arabidopsis thaliana
MAVPTLCIADGIYPTAFFYRNSVRRLTSVAHDRFTNPKFGIDGRLRNGPKVTATVNPYTYTEAAQPEERKSLTDFLVEARDFVRSDGGDGGPPRWFSPLECGARAPESPLLLYLPGIDGTGLGLIRQHKRLGEIFDIWCLHFPVTDRTPARDLVKLIERTVRSEYFRLPKRPIYIVGESIGACLALDVAASNPDIDLVLILANPVTRVNNFMLQPLSSLLEILPDGVPSFLEENFRFEQGYPFAAMFETMLNETDAAQIGGGLLGDLFATSVNLPTLARIFPKDTLLWKLQLLKSASASAKSHMYTVKAQTLILLSGRDQWLLNKEDIEKLHCTLPNCEVRKFENYGQLLFLEDGVDLVTIIKCTYYYRRGKLLDYVSDFILPTPFELKEYEESQRLLTAITSPVFLSTLDNGTVVRSLAGIPSEGPVLYVGNHMLLGTELRPAAIHFLKEKNILLRGLAHPVMFAKKYGSKLPDMHMFDSVRMIGAVPVSNINFYKLLRSKAHVVLYPGGVREALHRKGEVYKLFWPEHSEFVRTASKFGTKIIPFGVVGEDDLCEVVFDYNDQMKIPFLKNLIKELSQDSTYLRNGEEGEVGNQDLHMPGIVPKMPGRFYVYFGKPIYTEGREDELNDKEKAHEVYLQVKSEVERCMTYLKIKREGDPYRNILARSLYHFSHGFSSQVPTFDLRNQ

SEQ ID NO: 6 - protein, 684 aa, Arabidopsis thaliana
MAIITTTTVRFTDGTSPTFFSSASTKAYNLHFLYSNSTQRLTNPKFGIGGKLKVTVNPYSYTEEVRPEERKSLTDFLTEAGDFVNSDGGDGGPPRWFSPLECGARAPESPLLLYLPGIDGTGLGLIRQHKRLGEIFDIWCLHFPVKDRTPARDIGKLIEKTVRSEHYRFPNRPIYIVGESIGASLALDVAASNPDIDLVLILANPVTRFTNLMLQPVLALLEILPDGVPGLITENFGFYQEMFETMLNENDAAQMGRGLLGDFFATSSNLPTLIRIFPKDTLLWKLQLLKSASASANSQMDTVNAQTLILLSGRDQWLMNKEDIERLRGALPRCEVRELENNGQFLFLEDGVDLVSIIKRAYYYRRGKSLDYISDYILPTPFEFKEYEESQRLLTAVTSPVFLSTLKNGAVVRSLAGIPSEGPVLYVGNHMLLGMELHAIALHFLKERNILLRGLAHPLMFTKKTGSKLPDMQLYDLFRIIGAVPVSGMNFYKLLRSKAHVALYPGGVREALHRKGEEYKLFWPEHSEFVRIASKFGAKIIPFGVVGEDDLCEMVLDYDDQMKIPFLKNLIEEITQDSVNLRNDEEGELGKQDLHLPGIVPKIPGRFYAYFGKPIDTEGREKELNNKEKAHEVYLQVKSEVERCMNYLKIKRETDPYRNILPRSLYYLTHGFSSQIPTFDLRNH

SEQ ID NO: 7 - protein, 701 aa, Arabidopsis thaliana
MAVTVLPSVSGLSAVASSSNLRRLTSASNHRLTAIKSVTSTSSPPTPSSGVQRRRKNNDENRATVAKVVENPYSKVEAARPDLQKRLSDFLEEAREFVGDGGGPPRWFSPLECGAQATNSPLLLYLPGIDGTGLGLIRHHKKLGEIFDIWCLHIPVSDRTPVKDLVKLIEETVKSENFRLPNRPIYLVGESIGACLALDVAARNPNIDLSLILVNPATHVNNFMVQPLSGMLNVLPDGLPTLLEDIFDFGFKQGDPLTGMLDALSNEFSVQRMGGVGGGMLRDVLAVSANLPTLSRMFPKDTLLWKLEMLKYAIASVNSHIYSVRAETLILLSGRDHWLLKEEDIDRYSRTLPKCIVRKLDDNGQFPLLEDGVDLATIIKCTCFYRRGKSHDHITDYIMPTTFELKQQVDDHRLLMDGTSPVMLSTLEDGTVVRSLEGLPSEGPVLYVGYHMILGFELAPMVIQLMTERNIHLRGLAHPMLFKNLQDSLVDTKMFDKYKIMGGVPVSHFNIYKLLREKAHVLLYPGGVREALHRKGEEYKLFWPERSEFVRVASKFGAKIVPFGVVGEDDICEIVLDSNDQRNIPILKDLMEKATKDAGNIREGDESELGNQECYFPGLVPKIPGRFYYYFGKPIETAGKEKELKDKEKAQELYLQVKSEVEQCIDYLKVKRESDPYRHLLPRMLYQASHGWSSEIPTFDL

SEQ ID NO: 8 - protein, 692 aa, Hordeum vulgare
MSMVPHIMLRPFGVTPCGGGGGGCLRRHHARLCLPRASSVDATGGSEPGDGSNRRRRKKQAAEEPRLEVLYDDGFGSVTMKDYWEAVRAMPKDDGGPPRWFCPVECGRPEVDRSPLLLVLPGTDGVGMELILHHKSLGKVFEVCCFHIPLSDRTPFEGLLQIVEEYVKYESALSPSRPIYIIGDSFGGCLAISVAARNPEIDLVLTLVNPATSSAKTSLQAVLPLLEAVPSDLPVVHPHLLKYLIGNPLNVAMVSVQNGLSPQETLQEFSNSLASMLPLVSELGDIIQMGTLVWKLKLLKSGANYANSQLCAVQAEVLLLASGIENLPPSGEAHRLFKTLKNCKLRYFRNRGDRLLMEDGFNLLTVIKGVNMYRRGRQRDPVNDFLSPTLSEFKRTFGEDFKLFNQLLSPVMLSTLKNGNIVRGLAGVPDKGPVLFVGYHQLFAMEVLALIEGFLREKKTILRTTAHQVFFVGNYETLRQELSLFDWFSTYGALPVSPINTYKLFERNEFVLLYPGGVREALHRKGEAYKLFWPDQPEFVRMAARFGVTVIPFGCVGEDDFMEVVVDYNDQKNIPYLKDEIKSFNEDFTGIIRDTVKGDDGNQVLHVPAVLPKVPGRLYFLFGKPIEMKGMDNVLTDRKKANEVYLQIESEVENLMSYLKRKRSEDPYRSITRRALYQATRGPSTQVPTFEP

SEQ ID NO: 9 - protein, 682 aa, Oryza sativa
MSMSIALHAVLPPAAATPRRRPTRLRASSTEAPASGEKGQEDTDRKSGTGMRRRRRRAKKVQEVGLEALYDDGFGEATVRDYFDALRATPLDGGGGGGPPAVHAPPLLLFLPGIDGVGMELIMQHKSLGKVFEVRCLHIPVNDRTPYEGLLQIMEESVKYEHNLSPNRPIYIIGDSFGGCLALSLASRNPEIDLVLILVNPATSFAKTPLQAILPLLEMVPSNLPVTLPHLLRYLIGDPLKMAMVSIQNNTSPQDTLESFSDSLSSMLPLLSEFGHIVRMDTLVWKLKLLMSGVDYTNSRLNAVQAEILLLASGNDNLPPSGEADRLFKALKSCKVRYFRTSSDRLLMESSFNLLTVIKGASMYRQGKQRDTITDFLPPTISEFKRTFGEDFKLLHHLLSPVMLSTLRNGKIVRGLAGVPDKGPVLLVGYHQLLAMEITSMAEEFLREKKAVLRTLAHPVFFVGNYEILRQELSFFDVVPLYGGVQVSPINTYRLFERDEFVLLYPGGIREALHRKDEDYQLFWPDQPEFVRMAAQFGVTVIPFGCVGEDDMLEIVLDYNELKNIPYIRETIESFNQDCPGVRSTVKGEEGNQVLHLPAVLPKLPGRLYYLFGKPIEMKGMDGVQRDRESANQLYLDIKSEVENIMSYLKRKREQDPYRSITARTFYQATWGVTAQIPTFEP

SEQ ID NO: 10 - protein, 698 aa, Oryza sativa
MSAALHTLPAVAAGGLGCRWRRPSATLRAASSVGTASVAVSVNAETQQQRQVGVEEEEKRREDAAEVRTGRVVEALYDDGFGGVTVKDYFAAARAVSSDDGGPPRWFCPVDAGRPAVDNAPLLLFLPGTDGVGMGLILHHKSLGRVFEVRCLHIPVNDRTPFEGLLQIVENSIKYEHAMSPNRPIYLIGDSFGGCLALSVAARNPQIDLVLILINPATSFAKTPLQPILPVLEAMPSELHVTVPYLLSFVMGDPLKMAMVSVENNLSPPKTLQKLSDSLTSMLPLLSELADIIPRDTLFWKLKLLKSGAAYANSRLHAVQAEVLLLASGNDNLLPSGEEADRLFKSLKNCRVRYFKDNGHTLLLEDGVNLLSVIKGVNMYRRGRQRDPVTDYIPPTLSEFKKTFDEDHKLFHLALSPVMLSTLKNGKIVRGLTGVPDQGPVLFVGYHALMGIELSPLYEEFLREKRTSFRGMAHPILFGGKHESSRQELSRFDTISMYGGLPVTAINMYRLFERNQFVLLYPGGVREALHRKGEAYKLFWPDQPEFVRMAARFGVTIIPFGFVGEDDVLELVADYNDQKNIPYLREWIESINREAQRVRDSVKGEDGNQDVHIPALLPKVPGRFYYLFGKPIEMKGMDNVVRDRKSANEVYLHIKSEVESLMSYLKRKREEDPYRSIAQRAVYQASWGASAEVPTFEP

SEQ ID NO: 11 - protein, 664 aa, Oryza sativa
MATLSLPLHSQIALWRQRRRRHDLFERISSCHQFKHAGWRLQVSYKGLETLYDDGYQKAKDLDYYYRSLGELVEHDSGPPRLFCPVDAGSPIEDAPLMLYLPGVDGMGMGLFMHHKALGRIFELRCMHIPFHDRTPFEELVEMVEDVVRAEHATSPNKPIYLLGTSFGGCIALAVAARNPCIDLVLVLVNPATSFEKSDIKQLLSVSSPLSDRARIAITSLLNYNIDNEVDMALSSMKSGRHPLEALNRLTSNISSFLKHSNILNKIPEDTLGWKMKLIQQAASYANCRLESVSAEVLLLVSCADRLLPSKSEADRLQRMLPKCKVFFFENHGHSLLLEYGVHVSSIIKCTSLYRHSRRYHRVFDYIPPSATELKEVEKAGSDLRARTCPAMFSTMGDGVVVRGLAGVPEEGPVLLVGNHMLLGIELISLATEFLRRKGRVLRGIAHPLLFPNKTKTWSEGHDFFDFLNLWGGVPMTYKYIYQLLAAGEFVLLYPGGHREALHCKGEEHRLFWPDQTEFVRMAAQFNATIVPFGVVGEDDLMELLCTFEDIRNAPFGKEIMQAYSNHLKLRDIDHEVFFPGVYLKIPGRFYYRFGKPIPTKGMQAVMTDKQAAGELYLHVKSEVKAMIAYLLEKREEDKFRSILPRILYQLGCGHDSEIPSFDP

SEQ ID NO: 12 - protein, 665 aa, Oryza sativa
MPMPATVAAAPRPLLPGLPRRGTAPPRGRWSASAASAASRGVAAKAARRGVREYVEAAREMVRRPDGGPARWFSPLECGGGGGRLPGAPTMLYLPGIDGVGLGLIRHHERLAKMFDMWCLHIPVEDCTSFEGLVEYVESAVKSEGQRARDRPVYLVGESVGACIALAVAARNPDIDLVLILVNPGTSFHKSQLQSLSVFLDLVPEPFHLTTPQLLNFLTGNFMKIPSTIVGRGFSFQEAGQALSEITTSLLPSLMSLLDVLPKESIVWKLKMLRTASSFVNSRLHAVKAQTLVLASWNDELLPSREEAERLRDALEKCRIRNFKDNGHKILLEAEFDLATAIKGAGYYRRSLETDFVSDYLPLTPDEFQKATDHIRMLQYIANPVMLSTLPDGKIVRGLSGLPKQGPAVIVGYHMLLGFELGPLVTGVLRSSGIHIRGLAHPFMFDKKKEKIMPDPSYYDMHRIMGAVPVTAGNFYKLLAEKHFVLLYPGGAREALHRKGEEYKLFWPEQSEFVRMASRFGATIIPFGVVGEDDICDMLLDYDDLMKIPFYDILDRMLNEDGVKLRTDSTGELKYQRIHPVVAAPKIPGRFYFIFGKPIETRGREKELRDKENAQHLYLNVKSEVESCMKYLKEKREKDPYRNILARLLYQMVHGLDAEVPTFDP

SEQ ID NO: 13 - protein, 698 aa, Brassica rapa
MATTTTSSSLLALPSFRSNCNQRPSFKVRAQISGDKATSVEPVNGSVSVSNLPNPKGSEVNGKVKFQREAEIELLWDDGYGSKSVKDYFAAAREILKKPDGGPPRWFSPVDCGQPIKDAPTLFFLPGMDGTGMGLVPHQIALGKAFHVWCLHIPVHDRTPFQGLLKIVEDVLRQEHATRPNKPIYLVGDSFGGCMALAVAARNPSIDLVLVLVNPATSFERSPLQPLLPILEMVPEELHFTIPYALSFIMGDPIKMASIGIDNQLPAGVKLNKLREKLTNSMLPLLSELGGIIPRETLLWKLKLLRSGAAYTNSRIHAVEAEVLVLFSGKDRMLPSQEESKRLHGLLKNCTVRCFKENGHTLLLEDSLSLLTVIKGTCKYRRSRRYDFASDFLPPSKGELDFALQELLRFLRNAVSSVFLSTMEDGRIVKGLAGVPDEGPVLLVGYHMLMGLELGPMSEAFIKEKNILFRGMAHPILYAETDSTKEFGYMDWIKVFGAYPVTATNLFKLLSSKSHVLLYPGGAREALHNRGEQYKLIWPDQQEFVRMAARFGATIVPFGTVGEDDIAELVLDYNDLMKIPFLSNYITEATRDTKEFKLRDESSGEVANQQLYLPGVLPKIPGRFYYLFGKPIHTKGRPELVKDKEAAKLVYLEAKEEVEKCIAYLLKKREEDPYRSVVDRLSYSLTHLPENDVPSFEP

SEQ ID NO: 14 - protein, 706 aa, Populus trichocarpa
MTSVVSSQAMYCFFSTPEITPRFRVRVQSGSGGGNSKVSPSDSALVIGEKDKKFGGRIDSWNASLKCGIKKKSVKDVISSDLDVLWDDGYGTKTAKDFFEGAKEMIRPDGGPPRWFCPTECGQPLKDSPILLFCPGIVGVGLALTLHHKALGKVFEVRCLHIPVNDRTPFEGLVKFVEETVRLEHASSPNKPIYLVGDSFGGCLVLAVAARNPEIDLVVILANPATSFDRSQLRPLIPLWEALPDGLYNALPYLLSFVMGNPVEMARVNIEYRLPPRLQIKQLFQNLIALLPHLSDLVDIIPKDTLIWKLKLLKSAASYANSRLHAVKAEVLVLFSGNDHMLPSGDEAQRLKRTLKNCTVRYFKDNGHNILMEGGVNFLAVIKGTGKYRRSRRIDLVLDFIPPSMSEFKQGYDEVFGLLRFATGSAMFSTLNDGKIVKGLHGVPNEGPVLLVGNHMLMGLEIYSLVPEFLREKNIMVRVVVHPVVFRERQGVSSPEFSLADWMKVMGAVPVTASNLFNLLSTKSHVLLYPGGAREALHNRGEEYKLFWPDQQEFVRMAARFGATIVPFGTVGEDDVAELVLDYNDFMKIPVVNDYIRDANRNSIKLRDKSKGEVANQELYLPGILPKVPGRFYFLFGKPIETKDRKEEILEDRENANQLYLHIKSEVERCIAYLLKKREEDPYRSIVDRTVYRALHSPLHEVPAFD

SEQ ID NO: 15 - protein, 707 aa, Populus trichocarpa
MTSVVSSQAMYCFFSTPEITPRFRVRVQSGSGGGNSKVSPSDSALVIGEKDKKFGGRIDSWNASLKCGIKKKSVKDVISSDLDVLWDDGYGTKTAKDFFEGAKEMIRPDGGPPRWFCPTECGQPLKDSPILLFCPGIVGVGLALTLHHKALGKVFEVRCLHIPVNDRTPFEGLVKFVEETVRLEHASSPNKPIYLVGDSFGGCLVLAVAARNPEIDLVVILANPATSFDRSQLRPLIPLWEALPDGLYNALPYLLSFVMGNPVEMARVNIEYRLPPRLQIEQLFQNLIALLPHLSDLVDIIPKDTLIWKLKLLKSAASYANSRLHAVKAEVLVLFSGNDHMLPSGDEAQRLKRTLKNCTVRYFKDNGHNILMEGGVNLLTVIKGTGKYRRSRRIDLVLDFIPPSMSEFKQGYDEVIGLLRFATGSAMFSTLNDGKIVKGLHGVPNEGPVLLVGYHMLMGLEVYSLVPEFLREKNIMVRGVTHPVVFRERQGVSSPEFSLADWMKVMGAVPVTASNLFNLLSAKSHVLLYPGGAREALHNRGEEYKLFWPDQQEFVRMAARFGATIVPFGTVGEDDVAELVLDYNDFMKIPVINDYIRDANRNSIRLRDKSKGEVANQELYLPGILPKVPGRFYFLFGKPIETKDRKEEILEDRENANQLYLHIKSEVERCIAYLLKKREEDPYRSIVDRTVYRALHSPLHEVPAFDP

SEQ ID NO: 16 - protein, 723 aa, Populus trichocarpa
MASIFTCNISPYVALNSGNKPRFGVRAQSLGGGDSTVLSSDEIVVNGTSFVGPKEKNGALIDGGSGEKVKNGTLIDGGKNGRLKPTIEKKWVKDAVFKDLELLWDDGHGTKTVKDYLDGAKEIIKPDGGPPRWFCPAECGKPLKDSPVLLFLPGLDGVGLGLALHHKALGKAFEVRCLHIPVYDRTTFEGLVKIVEETVRLEHASSPNKPIYLVGESFGACLALAVAARNPKIDLVLILVNPATSFSRSQLPLLPILEALPDGLHDVFPYLVGFVTGNPVKMAMANIEYKLPPRLQFQQLYHNLTALLPSVSVLSDIIPKETLIWRLKLLKSAAAYANSRLHAVKAEVLVLASGNDNLLPSKDEAHRLKSSLKNCKVRFFKDNGHSMLMEDGLNLLTIIKGTCKYRRSRRLDLVSDFLPPSMSEFKCAFDEVTGLFRLATCSAVFSTLDDGKIVKGLAGVPDEGPVLFIGYHMLMGLEIYSLVDEFLREKNIMVRGVAHPDLFSEIMEGSSTEFSVSDWMKVMGAVPVTGSNLFKLLSKKSHVLLYPGGQREALHYKGEAYKLIWPDQPEFVRMAARFGATIVPFGTVGEDDIAELALDYHDLMKIPILNDFARDLMSKSSRVRDESKGEVASTDLFIPGLLPKIPGRFYFLFGKPIKTKGMKEMLEDKENAKQLYLHVKSEVQNSIAYLLKKREEDPYRSIIDRTIYRAFYSPLPEVPAFDP

SEQ ID NO: 17 - protein, 682 aa, Populus trichocarpa
MASFVTSGILPCFGLNSDVKPRFQARVQSSASVALKLGRLKPSIEEKKWVNDFGPKDLEPLWDDGYGTKTIKDYFDGAKEMIRPDGGPPRWFCPIECGQPLKSSPTLLFLPGVDGVGLGLTLHHKALGKAFEVLCLHIPVYDRTSFEGLVKNVEETVRLEHARNPNKPIYLVGESIGGCLAIAVAARNPKVDLVVILANPATSFGRSKLQPFLPILEAVPNQLHNGFLCLLSLLTGNPVKMAMVGVEDRLPSRLKIGKLYQNLMALLHNLSVVADIIPKDTLVWKMKLLRSAADYANSCLHNVKAEVLLLASEKDRLLPSRDEALRLKSLLQNCTVRNFKANGHAILLEDGVGLLTAIKGTSKYRRSKRIDFVSDYLPPSTSEFKSFFEEAYGLLLYAAGSTMFSTLEDGKIVKGLAGVPNEGPVLLVGYHMLMAFDIYPLGEGFLREKNIMVRGLGHPDLFTGKLEDSSNEFAYAEWIRVMGTVAGTASNLFKLLSTKSHVVLYPGGARESLHNKGEEYKLFWPDQQEFVRTAARFGATIVPFGTVGEDDLTHLVLDYHDMMKIPIVSDYIREVNSKATRIRDNSKGEVANQQVYIPGVLPKLPGRFYYLFGKPIKTKGMEDMLRDRENANQLYLHVKSEVENNIAYLLKKREEDPYRSLINRTIYQALHSPSSNVPTFDP

SEQ ID NO: 18 - DNA, 35 bp, Artificial Sequence
  Synthetic DNA
aaaaagcaggctcaatggcggatacagctagagga

SEQ ID NO: 19 - DNA, 36 bp, Artificial Sequence
  Synthetic DNA
ctcgcccttgctcaccatagtagtgtgctggccacc

SEQ ID NO: 20 - DNA, 36 bp, Artificial Sequence
  Synthetic DNA
ggtggccagcacactactatggtgagcaagggcgag

SEQ ID NO: 21 - DNA, 34 bp, Artificial Sequence
  Synthetic DNA
agaaagctgggtcttacttgtacagctcgtccat

SEQ ID NO: 22 - DNA, 29 bp, Artificial Sequence
  Synthetic DNA
ggggacaagtttgtacaaaaaagcaggct

SEQ ID NO: 23 - DNA, 28 bp, Artificial Sequence
  Synthetic DNA
ggggaccactttgtacaagaaagctggg

SEQ ID NO: 24 - DNA, 1239 bp, Artificial Sequence
  Synthetic DNA coding an Oleosin-GFP fusion gene
atggcggatacagctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactactatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaa

SEQ ID NO: 25 - protein, 412 aa, Artificial Sequence
  Synthetic Construct
MADTARGTHHDIIGRDQYPMMGRDRDQYQMSGRGSDYSKSRQIAKAATAVTAGGSLLVLSSLTLVGTVIALTVATPLLVIFSPILVPALITVALLITGFLSSGGFGIAAITVFSWIYKYATGEHPQGSDKLDSARMKLGSKAQDLKDRAQYYGQQHTGGEHDRDRTRGGQHTTMVSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK